SEQUENCE LISTING (12 sequences)

SEQ ID NO: 1 - DNA, 20 bp, Artificial sequence
  Synthetic polynucleotide
  MISC_FEATURE: AAVS1-1 sgRNA
ggggccactagggacaggat

SEQ ID NO: 2 - DNA, 20 bp, Artificial sequence
  Synthetic polynucleotide
  MISC_FEATURE: sgKM040 sgRNA
gatgccaaaaacacacatgt

SEQ ID NO: 3 - DNA, 18 bp, Artificial sequence
  Synthetic polynucleotide
  MISC_FEATURE: sgKM041 sgRNA
gaacaccggcccctgaag

SEQ ID NO: 4 - RNA, 20 nt, Artificial sequence
  Synthetic polynucleotide
  MISC_FEATURE: sgKM048 sgRNA
ccugccucaggaugaacuga

SEQ ID NO: 5 - RNA, 20 nt, Artificial sequence
  Synthetic polynucleotide
  MISC_FEATURE: sgKM049 sgRNA
uaaccagccugcugucucug

SEQ ID NO: 6 - DNA, 24 bp, Artificial sequence
  Synthetic polynucleotide
  MISC_FEATURE: oKM.061 primer
cacctaggacgcaccattctcaca

SEQ ID NO: 7 - DNA, 22 bp, Artificial sequence
  Synthetic polynucleotide
  MISC_FEATURE: oKM.062 primer
cttctccgacggatgtctccct

SEQ ID NO: 8 - DNA, 20 bp, Artificial sequence
  Synthetic polynucleotide
  MISC_FEATURE: oKM.160 primer
gagtgatgtttgagggcgcg

SEQ ID NO: 9 - DNA, 22 bp, Artificial sequence
  Synthetic polynucleotide
  MISC_FEATURE: oKM.161 primer
cgaccagtgtcaggaagggtat

SEQ ID NO: 10 - DNA, 25 bp, Artificial sequence
  Synthetic polynucleotide
  MISC_FEATURE: oKM.139 primer
caacaagttattgccactgctacgg

SEQ ID NO: 11 - DNA, 25 bp, Artificial sequence
  Synthetic polynucleotide
  MISC_FEATURE: oKM.140 primer
atcaggatgatgggagggaagacag

SEQ ID NO: 12 - RNA, 20 nt, Artificial sequence
  Synthetic polynucleotide
  MISC_FEATURE: sgKM046 sgRNA
ggauuuaaaguuucucucag

(19) United States
(12) Patent Application Publication
(10) Pub. No.: US 2025/0195650 A1
(43) Pub. Date: Jun. 19, 2025

(54) METHODS AND COMPOSITIONS FOR ENHANCING EFFICACY OF THERAPEUTIC IMMUNE CELLS

(71) Applicant: The Board of Trustees of the Leland Stanford Junior University, Stanford, CA (US)

(72) Inventors: Crystal L. MACKALL, Stanford, CA (US); Katherine Ann FREITAS, Palo Alto, CA (US); Elena SOTILLO-PINEIRO, Palo Alto, CA (US)

(21) Appl. No.: 18/251,696

(22) PCT Filed: Nov. 4, 2021
(86) PCT No.: PCT/US2021/058047

§ 371 Date: May 3, 2023

Related U.S. Application Data
(60) Provisional application No. 63/109,517, filed on Nov. 4, 2020.

(51) Int. Cl. A61K 40/11; A61K 40/31; A61K 40/42; A61P 35/00; C12N 5/0783; C12N 9/22; C12N 15/11; C12N 15/90

(52) U.S. Cl. CPC A61K 40/11; A61K 40/31; A61K 40/4215; A61K 40/4251; A61P 35/00; C12N 5/0636; C12N 9/22; C12N 15/11; C12N 15/907; C12N 2310/20; C12N 2501/51; C12N 2501/515

(57) ABSTRACT

The present disclosure generally relates to, inter alia, recombinant immune cells that have been engineered to express reduced levels of one or more subunits of the mediator complex, and particularly relate to engineered immune cells exhibiting enhanced effector functions. Also provided are methods for generating engineered immune cells with enhanced effector function, pharmaceutical compositions the same, as well as methods and kits for the prevention and/or treatment of a health condition in subjects in need thereof.

### CROSS REFERENCE TO RELATED APPLICATION

The present application claims priority to U.S. Provisional Patent Application Ser. No. 63/109,517, filed on Nov. 4, 2020, the disclosure of which is incorporated by reference herein in its entirety, including any drawings.

### STATEMENT REGARDING FEDERALLY SPONSORED R&D

This invention was made with Government support under contracts CA232568 and CA049605 awarded by the National Institutes of Health. The Government has certain rights in the invention.

### INCORPORATION OF THE SEQUENCE LISTING

This application contains a Sequence Listing, which is hereby incorporated herein by reference in its entirety. The accompanying Sequence Listing text file, named “078430-527001WO_Sequence Listing_ST25.txt,” was created on Oct. 5, 2021 and is 3.4 KB.

### FIELD

The present disclosure generally relates to, inter alia, recombinant immune cells that have been engineered to express reduced levels of one or more subunits of the mediator complex, and particularly relate to engineered immune cells having enhanced effector functions. Also provided are methods for generating engineered immune cells with an enhanced effector function, pharmaceutical compositions the same, as well as methods and kits for the prevention and/or treatment of a health condition in subjects in need thereof.

### BACKGROUND

Immune cells have the potential to target tumor cells while sparing normal tissues, and therefore immune cells can be potent and specific “living drugs.” Several clinical observations indicate that they can have major anti-cancer activity. For this reason, adoptive transfer of genetically modified immune cells has emerged as a potent therapy for various malignancies. For example, current modalities of adoptive T cell therapy include cells modified to express receptors specific for cancer antigens, such as chimeric antigen receptors (CARs) and high-affinity T cell receptors (TCRs). In adoptive T cell therapies, modified T cells are typically activated by exposure to the cognate antigen in vitro or ex vivo, expanded, and then administered to the individual, where they proliferate and exhibit cytolytic activity and/or send signals to initiate an immune response against the target cancer.

Recent developments using CAR modified autologous T cell (CART) therapy, which relies on redirecting T cells to a suitable cell-surface molecule on cancer cells such as B cell malignancies, have shown promising results in harnessing the power of the immune system to treat B cell malignancies and other cancers. For example, recent clinical trials using CAR-T cells specific for the CD19 protein expressed on B-cell malignancies demonstrated marked disease regression in a subset of patients with advanced cancers. This success has led to the FDA approval of two CD19-CAR T cell therapeutic agents, axicabtagene ciloleucel (YESCARTA®) and tisagenlecleucel (KYMRIAH®), and other therapies in clinical development of medication for the treatment of large B-cell lymphoma (LBCL) and B-cell acute lymphoblastic leukemia (B-ALL).

However, extending this therapy to other types of cancers, especially to solid tumors poses several challenges. For example, in contrast to hematologic malignant cells, such as B-cells express CD19 where almost tumor-exclusive antigen to target, which allows specificity and therefore a wide therapeutic window, solid tumors usually reside in not readily-accessible sites via lympho-vascular circulation, isolated by dense stroma and tumor microenvironment which harbor immunosuppressive leukocytes and cytokines. Barriers against migration of cytotoxic T cells (CTLs) also include preference to non-target organs such as lungs, liver and spleen, limited lymphocyte extravasation due to oncotic pressure caused by the abnormal vascular formation, downregulated expression of adhesion molecules on tumor vasculature and reduced release of lymphocyte-attracting chemokines. Furthermore, tumor heterogeneity in solid tumors poses a challenge against antigen selection. Additionally, the tumor microenvironment elicits a number of tolerance and immunosuppression mechanisms that can reduce the effectiveness of adoptive cell therapies. Furthermore, besides the ability for the CAR-T cells to recognize and destroy the targeted cells, a successful therapeutic T cell therapy needs to have the ability to proliferate, to persist over time, and to further monitor for cancer cell escapees. The variable phenotypic state of T cells, whether it is in a state of anergy, suppression or exhaustion, have been reported to have various effects on CAR-T cells' efficacy. In addition, to be effective, CAR-T cells need to persist, e.g., survive in vivo after administration, and maintain the ability to proliferate in response to the CAR's antigen.

Thus, new compositions and strategies are needed for generating improved therapeutic cells for adoptive cell therapy. The presently disclosed aspects and embodiments address these needs and provide other related advantages.

### SUMMARY

Provided herein, inter alia, are novel methods and compositions for the prevention and/or treatment of various health conditions. In particular, described herein are engineered immune cells having enhanced therapeutic efficacy for, e.g., cancer therapy. Some embodiments of the disclosure relate to immune cells that have been engineered to express reduced levels of one or more subunits of the mediator complex. In some embodiments, the engineered immune cells exhibit enhanced effector functions. Also provided are methods for generating a population of engineered immune cells with an enhanced effector function, and pharmaceutical compositions containing such a population of engineered immune cells with enhanced effector function, as well as methods and kits for the prevention and/or treatment of a health condition in subjects in need thereof.

In one aspect, provided herein are methods for generating an engineered immune cell with enhanced effector function, the method including introducing into the immune cell a nucleic acid and/or a polypeptide capable of reducing expression level of a mediator complex subunit in the immune cell.

Non-limiting exemplary embodiments of the disclosed methods can include one or more of the following features. In some embodiments, the mediator complex subunit is selected from the middle module subunits, the tail module subunits, and the cyclin-dependent-kinase 8 (CDK8) module subunits of the mediator complex. In some embodiments, the mediator complex subunit is selected from the group consisting of CCNC, CDK8, CDK19, MED12, MED12L, MED13, MED13L, MED19, MED24, and MED26. In some embodiments, the mediator complex subunit is a middle module subunit. In some embodiments, the middle module subunit is MED19 or MED26. In some embodiments, the mediator complex subunit is a tail module subunit. In some embodiments, the tail module subunit is MED15, MED16, or MED24. In some embodiments, the mediator complex subunit is a CDK8 module subunit. In some embodiments, the CDK8 module subunit is selected from the group consisting of CCNC, CDK18, CDK19, MED12, MED12L, and MED13. In some embodiments, the nucleic acid is incorporated into one or more of the following: (i) a guide RNA (gRNA) of a CRISPR/Cas genome editing system, (ii) a TALEN (transcription activator-like effector nuclease) genome editing system, (iii) a DNA-guided endonuclease genome editing with NgAgo (Natronobacterium gregoryi Argonaute), (iv) anti-sense nucleic acid molecule, (v) a double-stranded RNAi molecule, or (vi) a hairpin-RNA molecule capable of inducing suppression or degradation of mRNA. In some embodiments, the nucleic acid includes a polynucleotide sequence having sufficient sequence complementarity to a target sequence within an endogenous genomic locus encoding the mediator complex subunit.

In some embodiments, the immune cell is T lymphocyte, a natural killer (NK) cell, or a natural killer T cell (NKT). In some embodiments, the T lymphocyte is a CD8+T cytotoxic lymphocyte cell selected from the group consisting of naïve CD8+ T cells, central memory CD8+ T cells, effector memory CD8+ T cells, effector CD8+ T cells, CD8+ stem memory T cells, bulk CD8+ T cells. In some embodiments, the T lymphocyte is a CD4+T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells.

In some embodiments, the methods of the disclosure further include introducing into the immune cell one or more recombinant immune receptors, such as a chimeric antigen receptor (CAR) or a T cell receptor (TCR).

In one aspect, provided herein are engineered immune cells including a nucleic acid and/or a polypeptide capable of reducing expression level of a mediator complex subunit in the immune cell. In some embodiments, the mediator complex subunit is selected from the middle module subunits, the tail module subunits, and the cyclin-dependent-kinase 8 (CDK8) module subunits of the mediator complex. In some embodiments, the mediator complex subunit is selected from the group consisting of CCNC, CDK8, CDK19, MED12, MED12L, MED13, MED13L, MED19, MED24, and MED26. In another aspect, provided herein are engineered immune cells produced by a method of the disclosure. Non-limiting exemplary embodiments of the engineered immune cells described herein can include one or more of the following features. In some embodiments, the immune cell is in vitro, ex vivo, or in vivo. In some embodiments, the immune cell is a T lymphocyte. In some embodiments, the immune cell is an exhausted immune cell or a non-exhausted immune cell. In a related aspect, provided herein cell cultures including at least one engineered immune cell of the disclosure, and a culture medium.

In another aspect, provided herein are pharmaceutical compositions including a pharmaceutically acceptable excipient and a) an engineered immune cell of the disclosure; and/or b) a nucleic acid including a sequence having sufficient sequence complementarity to a target sequence within a genomic locus encoding a mediator complex subunit, wherein the mediator complex subunit is selected from the middle module subunits, the tail module subunits, and the cyclin-dependent-kinase 8 (CDK8) module subunits of the mediator complex. In some embodiments, the mediator complex subunit is selected from the group consisting of CCNC, CDK8, CDK19, MED12, MED12L, MED13, MED13L, MED19, MED24, and MED26.

Non-limiting exemplary embodiments of the pharmaceutical compositions described herein can include one or more of the following features. In some embodiments, the composition includes at least one engineered immune cell of the disclosure, and a pharmaceutically acceptable excipient. In some embodiments, the composition includes a nucleic acid including a sequence having sufficient sequence complementarity to a target sequence within a genomic locus encoding a mediator complex subunit, wherein the mediator complex subunit is selected from the middle module subunits, the tail module subunits, and the cyclin-dependent-kinase 8 (CDK8) module subunits of the mediator complex. In some embodiments, the mediator complex subunit is selected from the group consisting of CCNC, CDK8, CDK19, MED12, MED12L, MED13, MED13L, MED19, MED24, and MED26. In some embodiments, the composition including the nucleic acid is encapsulated in a viral capsid, a liposome, or a lipid nanoparticle (LNP).

In another aspect, provided herein are methods for treating a health condition in a subject in need thereof, the method including administering to the subject a composition including: (a) an engineered immune cell of the disclosure; b) a nucleic acid including a sequence having sufficient sequence complementarity to a target sequence within a genomic locus encoding a mediator complex subunit, wherein the mediator complex subunit is selected from the middle module subunits, the tail module subunits, and the cyclin-dependent-kinase 8 (CDK8) module subunits of the mediator complex. In some embodiments, the mediator complex subunit is selected from the group consisting of CCNC, CDK8, CDK19, MED12, MED12L, MED13, MED13L, MED19, MED24, and MED26; and/or c) a pharmaceutical composition of the disclosure.

Non-limiting exemplary embodiments of the treatment methods described herein can include one or more of the following features. In some embodiments, the health condition is a proliferative disease, an autoimmune disease, or an infection. In some embodiments, the subject is a mammalian subject. In some embodiments, the mammalian subject is a human subject. In some embodiments, the subject has or is suspected of having a proliferative disease, an autoimmune disease, or an infection. In some embodiments, the proliferative disease is a cancer. In some embodiments, the cancer is a leukemia or an osteosarcoma. In some embodiments, the administered composition confers enhanced effector function selected from the group consisting of growth rate (proliferation), cytokine production, target cell inhibition (e.g., anti-cancer cytotoxicity), macrophage activation, T cell activation, NK cell activation, and in vivo persistence (e.g., survival). In some embodiments, the enhanced effector function includes increased production of interferon gamma (INFY), interleukin-2 (IL-2), and/or tumor-necrosis factor α (TNFα). In some embodiments, the enhanced effector function comprises increased effector memory T cell phenotype. In some embodiments, the enhanced effector function comprises oxygen consumption and extracellular acidification rate. In some embodiments, the composition is administered to the subject individually (monotherapy) or as a first therapy in combination with a second therapy, wherein the second therapy is selected from the group consisting of chemotherapy, radiotherapy, immunotherapy, hormonal therapy, toxin therapy, or surgery.

In yet another aspect, provided herein are kits for the prevention and/or treatment of a condition in a subject in need thereof, the kit including: (a) an engineered immune cell of the disclosure; (b) a nucleic acid including a sequence having sufficient sequence complementarity to a target sequence within an endogenous genomic locus encoding a mediator complex subunit, wherein the mediator complex subunit is selected from the middle module subunits, the tail module subunits, and the cyclin-dependent-kinase 8 (CDK8) module subunits of the mediator complex; and/or c) a pharmaceutical composition of the disclosure. In some embodiments, the mediator complex subunit is selected from the group consisting of CCNC, CDK8, CDK19, MED12, MED12L, MED13, MED13L, MED19, MED24, and MED26.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 schematically depicts the generation of CAR-T cell CRISPR knock-out library. T cells were purified from two human donors. A CRISPR library targeting all ˜20,000 protein coding genes with 10 guides per gene was integrated into 200 million T cells at low multiplicity of infection (10% positive) using lentiviral vector. Purified Cas9 protein was electroporated into T cells on Day 3, and CAR was integrated by retrovirus on Days 3 and 4 post activation.

FIG. 2 schematically depicts a CRISPR screen experimental design. Gene edited CAR-T cells were cultured in vitro for 2 weeks. Expression of a tonic signaling CAR induces progressive T cell dysfunction. To screen for cytokine production, a fraction of the population was stimulated with tumor cells and sorted by FACS for T cells that express high levels of IL-2 and TNFα. To screen for proliferation, T cells were co-cultured with tumor cells expressing the CAR-T target for 7 more days.

FIG. 3 graphically summarizes the results of experiments performed to illustrate that selected genes from proliferation screen demonstrate reproducibility between replicate donors. The abundance of guide RNAs was quantified at Day 0 and at Day 23. The plot shows the average log 2 (fold change) of all guide RNAs targeting each gene. Genes plotted in the upper right quadrant enhance proliferation when deleted, and genes in the lower left quadrant decrease proliferation when deleted.

FIGS. 4A-4B graphically summarize the results of experiments performed to illustrate a genome-wide CRISPR screen that identifies statistically significant genes. The MAGECK algorithm was used to analyze the relative abundance of guides and calculate adjusted P values for each gene (Li 2014). As shown in FIG. 4A, the cytokine production screen compares guide abundance in the total Day 15 population to the cytokine high population. As shown in FIG. 4B, the proliferation screen compares abundance on Day 23 to Day 0. Guides targeting safe, non-coding regions of the genome were included as controls and the average log 2 (fold change) for the safe targeting guides is indicated by the vertical dashed line. The threshold for statistical significance is indicated by the horizontal dashed line. The cytokine production screen identified 1 statistically significant gene, while the proliferation assay identified several statistically significant genes.

FIGS. 5A-5B graphically summarize the results of experiments performed to illustrate that all 33 Mediator complex subunits were detectable in proliferation screen. Average log 2 (fold change) for all guides targeting each gene is plotted. First, an average enrichment for each guide was calculated by averaging donor 1 and donor 2. Then, the average enrichment for each gene was calculated by averaging the guide averages. Error bars depict standard deviation of the guides. MED12 and CCNC are both found in the CDK8 kinase module (CKM). Loss of all members of the CKM enhanced proliferation, with the exception of MED12L, which is not expressed in T cells. Loss of Mediator subunits found in the head, backbone, and middle domains reduced proliferation, with the exception of MED26 and MED19, which were anticipated to form the physical contacts between the middle domain and CKM. Additionally, a few members of the tail region (e.g., MED27, MED15, MED16, and MED24) slightly enhanced proliferation when deleted.

FIGS. 6A-6F graphically summarize the results of experiments performed to illustrate that MED12-null and CCNC-null CAR T cells produce more IL-2 and IFNγ. Target genes CCNC and MED12 and control gene AAVS1 were deleted on Day 3 post-activation. CAR-T cells were generated using the CD19-28ζ, HA-28ζ, and HER2-4-1BBζ receptors, cultured until Day 10 or 15, and subsequently co-cultured with NALM6, NALM6-GD2, or 143B respectively. Supernatants were collect 24 hours after the addition of tumor cells. Cytokines were quantified by ELISA. Mock-transduced T cells do not express a CAR and were included for a negative control. The bar graphs depict averages of two technical replicates, and error bars show the standard deviation.

FIG. 7 graphically summarizes the results of experiments performed to demonstrate that MED12-null CAR-T cells produce more IL-2 and TNFα on a single-cell basis. CD19-28ζCAR-T cells were stimulated with NALM6 tumor cells on Day 15 in the presence of monensin for 6 hours. Unstimulated (upper panel) and stimulated cell (lower panel) were fixed and stained for IL-2 and TNFα and analyzed by flow cytometry. The percentage of IL-2+ TNFα+ cells out of total CD4+ cells is displayed above each plot.

FIG. 8 graphically summarizes the results of experiments performed to demonstrate that MED12-null and CCNC-null CD19-28ζ CAR-T cells proliferate more in culture. Target genes CCNC and MED12 and control gene AAVS1 were deleted on Day 3 post-activation and cells were cultured with IL-2 until Day 28. Average total live cells counts are plotted and error bars depict standard deviation of three technical replicates.

FIG. 9 graphically summarizes the results of experiments performed to demonstrate that MED12-null and CCNC-null CD19-28ζ CAR-T cells are dependent on IL-2 for survival. On Day 9, cells were washed and plated in media with or without IL-2. Cell density was maintained between 0.5 and 1 million cells per mL medium. Cells were stained with acridine orange and propidium iodide and viability was monitored with the CellacaMX cell counter. In the absence of IL-2, no viable cells were detected in any condition by Day 28.

FIGS. 10A-10C graphically summarize the results of experiments performed to illustrate that MED12-null and CCNC-null CD19-28ζ CAR-T cells demonstrate increased tumor clearance in vivo. 1 million NALM6 cells expressing a luciferase transgene were infused on Day 0, and 250,000 CAR-T cells or mock transduced T cells were infused on Day 3. Tumor burden was monitored by bioluminescence imaging using the SII Lago (Spectral Instruments Imaging) and photons per second (p/s) were quantified with Aura Imaging software. 30 second exposures are shown in FIG. 10A, and shorter exposures were used for quantification to avoid saturation (FIGS. 10B and 10C). Values for individual mice are shown in B and averages are show in C. Error bars depict standard deviation.

FIG. 11 graphically summarizes the results of experiments performed to illustrate that MED12-null and CCNC-null CD19-28ζ CAR-T cells demonstrate increased expansion in vivo. Blood was collected from mice (as shown in FIG. 10) ten days after T cell infusion. Blood was mixed with an equal volume of CountBright Absolute Counting Beads (Invitrogen), stained for human CD45, and red blood cells were lysed with BD FACS Lysing Solution (BD). Human CD45+ cells were quantified by flow cytometry.

FIG. 12 graphically summarizes the results of experiments performed to illustrate that MED12-null CD19-28ζ CAR-T increase survival benefit of CAR-T cell treatment. Mice were infused with 1 million NALM6 tumor cells and treated with 1×10⁵, 2.5×10⁵, or 5×10⁵ T cells on Day 3. MED12-null CAR-T cells increased survival, while CCNC-null CAR-T cells were equivalent to AAVS1-null CAR-T cells.

FIG. 13 graphically summarizes the results of experiments performed to illustrate that MED12-null and CCNC-null HER2-4-1BBζ CAR-T cells decrease solid tumor growth and increase survival benefit of CAR-T cell treatment. 1 million 143B osteosarcoma cells were injected intramuscular on Day 0 and 5 million CAR-T cells or mock-transduced T cells were infused on Day 4. Solid tumors were measured by calipers. Mice were euthanized when tumor diameter was ≥17 mm.

FIG. 14 graphically summarizes the results of experiments performed to illustrate that gene deletion frequency is increased by targeting 2 cut sites in MED12 exon 2. Alt-R® S.p. Cas9 Nuclease V3 (IDT) was diluted to 5 mg/mL in Duplex Buffer (IDT). sgRNAs (Synthego) were resuspended at 100 μM in TE buffer. 1 μL CAS9 and 1 μL sgRNA was combined and incubated 30 minutes at room temperature. For the two guide condition, 0.5 μl of each sgRNA was added. 2 million T cells were resuspended in 18 μL P3 buffer, mixed with CAS9, and pulsed with protocol EO115 using the P3 Primary Cell 4D-Nucleofector™ S Kit and 4D-Nucleofector™ System (Lonza). Cells were recovered immediately with 100 μL warm media, then transferred to 1 mL warm media for 6 hours prior to transduction with CAR. Editing was performed on Day 3 and gene deletion was assessed on Day 11.

FIGS. 15A-15B graphically summarize the results of experiments performed to illustrate that loss of MED12 or CCNC increases cytokine production in CAR-T cells or non-transduced T cells.

FIGS. 16A-16B graphically summarize the results of experiments performed to illustrate that loss of MED12 increases expansion of CAR-T cells and T cells that do not express a CAR.

FIG. 17 graphically summarizes the results of experiments performed to illustrate that loss of MED12 or CCNC increases expansion in vivo.

FIGS. 18A-18B graphically summarize the results of experiments performed to illustrate that treatment with CCNC-null HER2-4-1BBζ CAR T cells reduces tumor area and increases survival of CAR-treated mice.

FIGS. 19A-19D graphically summarize the results of experiments performed to illustrate that survival of CAR-treated mice is increased after treatment with CCNC-null HER2-4-1BBζ CAR-T cells.

FIGS. 20A-20B graphically summarize the results of experiments performed to illustrate that the loss of MED12 increases expression of IL2RA in T cells.

FIGS. 21A-21B graphically summarize the results of experiments performed to illustrate that the loss of MED12 increases effector memory T cell phenotype. SCM: stem cell-like memory T cells; CM: central memory T cells; EM: effector memory T cells; TE: terminally differentiated T cells.

FIG. 22 graphically summarizes the results of experiments performed to illustrate that a deficiency in MED12 increases the oxygen consumption rate and the extracellular acidification rate in CD19-28z CAR-T cells 15 days post activation.

### DETAILED DESCRIPTION OF THE DISCLOSURE

The present disclosure generally relates to, inter alia, methods and compositions for the prevention and/or treatment of various health conditions. In particular, described herein are immune cells that have been engineered to express alleviated levels of one or more subunits of the mediator complex, and particularly relate to engineered immune cells exhibiting enhanced effector functions. Also provided are methods for generating a population of engineered immune cells with enhanced effector function, pharmaceutical compositions the same, as well as methods and kits for the prevention and/or treatment of a health condition in subjects in need thereof.

Recent developments using CAR-T cell therapy, which relies on redirecting T cells to a suitable cell-surface molecule on cancer cells such as B cell malignancies, have shown promising results in harnessing the power of the immune system to treat B cell malignancies and other cancers. In adoptive T cell therapies, modified T cells are typically activated by exposure to the cognate antigen in vitro or ex vivo, expanded, and then administered to the individual, where they proliferate and exhibit cytolytic activity and/or send signals to initiate an immune response against the target cancer.

Experimental data presented herein demonstrate that by down-regulating expression and/or activity of a mediator complex subunit, for example MED12 or CCNC, in immune cells leads to enhanced immune cell activation, cytokine secretion and tumor killing. In particular, when genes encoding subunits of the cyclin dependent kinase module (CKM) of the mediator complex were genetically disrupted in T cells, the genetically modified T cells were more proliferative, produced more inflammatory cytokines, and demonstrated increased anti-cancer cytotoxicity. As described in greater detail below, genes that exhibit this effect when deleted include CCNC, MED12, MED13, CDK8, and CDK19. Additionally, the experimental results described herein have demonstrated that deleting the genes encoding mediator complex subunits that form the physical contacts between the CKM and the core mediator complex (MED19 and MED26) yields the same effect on T cell function.

The findings described in the present disclosure can be of great value in the context of adoptive immunotherapy, where a specific receptor engagement is required such as in CAR immune cell therapy (including T cells, NK cells and NKT cells), TCR-modified T cell or tumor infiltrating lymphocytes (TILs) and where tumor-reactive cells compete for nutrients with tumor cells. In particular, the approach described herein may be particularly valuable for the treatment of solid tumors where a hostile tumor microenvironment (TME) with limited nutrients is documented. In addition, this approach could be applied to increase proliferation and expansion of immune cell products throughout the manufacturing process.

As discussed in greater detail below, deletion of gene(s) encoding mediator complex subunits could be accomplished with CRISPR/Cas system, or with homologous recombination, or any other genetic engineering method. Human primary T cells with any of the these genetic modifications can be transformed with a chimeric antigen receptor (CAR) or native T cell receptor (TCR) to create CAR T cells that can be subsequently used to treat human cancers. Alternatively, these genetic modifications could be made in tumor infiltrating lymphocytes (TILs) that are collected from patient tumors, expanded ex vivo, and reinfused into cancer patients. These genetic changes may also be useful in other lymphocytes such as natural killer cells or macrophages which are also used for adoptive cell therapy. Stated differently, the approaches described herein indicate that immune cells expressing natural receptors or those engineered to express antigen specific receptors such as chimeric antigen receptors (CARs), recombinant TCRs or others can be metabolically reprogrammed by down-regulation of one or more mediator complex subunits in order to improve their cytotoxic function, proliferation and in vivo persistence.

Furthermore, without being bound to any particular theory, it is contemplated that inhibiting the catalytic function of the mediator complex through pharmacological inhibition of CDK8 or CDK19 can result in a similar effect to genetic deletion of genes encoding subunits of the cyclin-dependent-kinase 8 module (CKM). Additionally, mutating amino acids within the catalytic domains of CDK8 or CDK19, thereby abolishing kinase activity, is contemplated to produce an effect similar that caused by loss of CKM. Moreover, other methods to diminish the function of the CKM such as RNAi knockdown of the aforementioned genes, or overexpression or additional genetic engineering of the core mediator subunit could produce the same effect. For example, protein engineering of subunits within the middle domain of the mediator complex to abrogate association of the CKM with the core mediator complex is anticipated to cause the same effect as that caused by loss of CKM. Such protein engineering could be accomplished by mutating amino acids that form the contacts between the CKM and core mediator. The experimental data presented herein indicate the key points of contacts are within MED26 and MED19. The term “inhibition” includes partial and complete inhibition. For example, the catalytic function of the mediator complex can be inhibited by at least about 20%, at least about 30%, at least about 40%, at least about 50%, at least about 60%, at least about 70%, at least about 80%, or at least about 90% by one or more pharmacological approaches, compounds, or means.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values. In some embodiments, the term “about” indicates the designated value ±up to 10%, up to ±5%, or up to ±1%.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

“Cancer” refers to the presence of cells possessing several characteristics typical of cancer-causing cells, such as uncontrolled proliferation, immortality, metastatic potential, rapid growth and proliferation rate, and certain characteristic morphological features. Cancer cells can aggregate into a mass, such as a tumor, or can exist alone within a subject. A tumor can be a solid tumor, a soft tissue tumor, or a metastatic lesion. As used herein, the term “cancer” also encompasses other types of non-tumor cancers. Non-limiting examples include blood cancers or hematological cancers, such as leukemia. Cancer can include premalignant, as well as malignant cancers.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that, operably linked elements may be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid molecules disclosed herein may be contiguous or non-contiguous (e.g., linked to one another through a linker).

The term “recombinant” or “engineered” nucleic acid molecule, polypeptide, or cell as used herein, refers to a nucleic acid molecule, polypeptide, or cell that has been altered through human intervention.

As used herein, and unless otherwise specified, a “therapeutically effective amount” or a “therapeutically effective number” of an agent is an amount or number sufficient to provide a therapeutic benefit in the treatment or management of a disease, e.g., cancer, or to delay or minimize one or more symptoms associated with the disease. A therapeutically effective amount or number of a compound means an amount or number of therapeutic agent, alone or in combination with other therapeutic agents, which provides a therapeutic benefit in the treatment or management of the disease. The term “therapeutically effective amount” can encompass an amount or number that improves overall therapy of the disease, reduces or avoids symptoms or causes of the disease, or enhances therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human subject) and non-human animals. In some embodiments, a “subject” or “individual” is a patient under the care of a physician. Thus, the subject can be a human patient or a subject who has, is at risk of having, or is suspected of having a disease of interest (e.g., cancer) and/or one or more symptoms of the disease. The subject can also be a subject who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Mediator Complex

The mediator complex is a multi-subunit assembly that has been reported to be required for regulating expression of most RNA polymerase II (pol II) transcripts, which include protein-coding and most non-coding RNA genes. Mediator and pol II function within the pre-initiation complex (PIC), which consists of mediator complex, pol II, TFIIA, TFIIB, TFIID, TFIIE, TFIIF and TFIIH. Mediator serves as a central scaffold within the PIC and helps regulate pol II activity in ways that remain poorly understood. Mediator is also generally targeted by sequence-specific, DNA-binding transcription factors (TFs) that work to control gene expression programs in response to developmental or environmental cues. At a basic level, mediator complex functions by relaying signals from TFs directly to the pol II enzyme, thereby facilitating TF-dependent regulation of gene expression.

Therefore, mediator is believed to be essential for converting biological inputs (communicated by TFs) to physiological responses (via changes in gene expression). For this reason, the mediator complex is considered a global regulator of gene expression and as such, is considered a general transcription factor. However, what distinguishes mediator from other general transcription factors (with the possible exception of TFIID) is its high degree of structural flexibility, its variable subunit composition, and its general requirement for activated (e.g., enhancer driven) transcription. Consistent with its ability to stimulate activated transcription, mediator appears to be the main binding interface for DNA-binding TFs within the PIC. These features are important for both general and context-specific functions, such that this “general transcription factor” may operate in mechanistically distinct ways at different genes or in different cell types.

### Mediator Subunits and Modules

The mediator complex is composed at least 31 subunits in all eukaryotes studied: MED1, MED4, MED6, MED7, MED8, MED9, MED10, MED11, MED12, MED13, MED13L, MED14, MED15, MED16, MED17, MED18, MED19, MED20, MED21, MED22, MED23, MED24, MED25, MED26, MED27, MED28, MED29, MED30, MED31, CCNC, and CDK8. For example, compositionally distinct forms of human mediator can be isolated as stable entities, with the most common being a 26 subunit “core” complex (21 subunit in Saccharomyces cerevisiae) and a 29 subunit “CDK8-mediator” complex (25 subunit in S. cerevisiae). The subunit composition of the human core mediator complex includes MED1, MED4, MED6, MED7, MED8, MED9, MED10, MED11, MED14, MED15, MED16, MED17, MED18, MED19, MED20, MED21, MED22, MED23, MED24, MED25, MED26, MED27, MED28, MED29, MED30, and MED31. The subunit composition of the human CDK8-mediator complex includes CDK8, CCNC, MED12, and MED13. In addition, there are three fungal-specific components, referred to as Med2, Med3 and Med5.

Structurally, mediator can be divided onto 4 main parts: the head, middle, tail, and the transiently associated CDK8 kinase module. The head and the middle modules interact directly with RNA polymerase II, whereas the elongated tail module interacts with gene-specific regulatory proteins. Mediator containing the CDK8 module is less active than mediator lacking this module in supporting transcriptional activation. The head module contains MED6, MED8, MED11, SRB4/MED17, SRB5/MED18, SRB2/MED20 and SRB6/MED22. The middle module contains: MED1, MED4, NUT1/MED5, MED7, CSE2/MED9, NUT2/MED10, ROX3/MED19, SRB7/MED21, MED26, and SOH1/MED31. CSE2/MED9 interacts directly with MED4. The tail module contains: MED2, PGD1/MED3, MED5, GAL11/MED15, SIN4/MED16, MED23, MED24, MED25, MED27, MED28, and MED30. The backbone is composed of MED14. The CDK8 module contains: MED12 (or MED12L), MED13 (or MED13L), CCNC, and CDK8 (or CDK19). In addition, individual preparations of the Mediator complex lacking one or more distinct subunits have been variously termed ARC, CRSP, DRIP, PC2, SMCC and TRAP. Additional information regarding mediator complexes in human and other eukaryotes can be found in, for examples, reviews by Poss Z. C. et al. (The Mediator complex and transcription regulation. Crit Rev Biochem Mol Biol. 2013 December; 48 (6): 575-608) and by Allen B. L. and Taatjes D. J. (The Mediator complex: a central integrator of transcription. Nat Rev Mol Cell Biol. 2015 March; 16 (3): 155-166), both which are herein incorporated by reference.

Methods for Generating Engineered Immune Cells with Enhanced Effector Functions.

As described in greater detail herein, some embodiments of the present disclosure provide various methods for generating an engineered immune cell with enhanced effector function, the method including introducing into the immune cell a nucleic acid and/or a polypeptide capable of modulating level of one or more mediator complex subunits in the immune cell. The term “modulating”, in relation to the level of a mediator complex subunit refers to a change in level of expression (e.g., transcription and/or translation), level of at least one biological activity of the mediator complex subunit (e.g., binding to its natural ligands). Modulation includes both increase (e.g., induce, stimulate) and decrease (e.g., reduce, inhibit), or otherwise affecting the level of the mediator complex subunit. For example, it has been reported that MED1 overexpression can results in increased expression of JUN, EGFR, and other proliferation associated genes. MED1 is overexpressed in 50% of breast cancers. Overexpression of MED20 and MED31 is described in osteosarcoma, further supporting the notion that overexpression of some subunits may increase proliferation. In some embodiments, the method includes introducing into the immune cell a nucleic acid and/or a polypeptide capable of inducing expression level of one or more mediator complex subunits in the immune cell. In some embodiments, the method includes introducing into the immune cell a nucleic acid and/or a polypeptide capable of inducing MED1, MED20, MED31, or a combination of any thereof. In some embodiments, the method includes introducing into the immune cell a nucleic acid and/or a polypeptide capable of reducing (e.g., alleviating) expression level of one or more mediator complex subunits in the immune cell. Non-limiting exemplary embodiments of the disclosed methods can include one or more of the following features. In some embodiments, the method including introducing into the immune cell a nucleic acid and/or a polypeptide that results in reduced expression level (e.g., alleviated expression) of one or more endogenous genes encoding one or more mediator complex subunits in the immune cell. In some embodiments, the introduced nucleic acid and/or a polypeptide results in a reduced expression level of one or more mediator complex subunits by at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% compared to a control (e.g., non-engineered immune cell or untransduced immune cell). In some embodiments, the introduced nucleic acid and/or a polypeptide results in about 95%, about 96%, about 97%, about 98%, or about 99%, or about 100% compared to a control (e.g., non-engineered immune cell or untransduced immune cell). Examples of immune cells having 100% reduction in expression level of a mediator complex subunit include engineered immune cells wherein the endogenous gene encoding the mediator complex subunit has been knocked-out or deleted (e.g., null mutant. See also, e.g., Examples 3-12.

Suitable mediator complex subunits include, but are not limited to, mediator complex subunits belonging to a core mediator complex, a CDK8-mediator module, a head module, a middle module, or a tail module. In some embodiments, the method including introducing into the immune cell a nucleic acid and/or a polypeptide capable of reducing expression level of a subunit of the core mediator complex, such as, MED1, MED4, MED6, MED7, MED8, MED9, MED10, MED11, MED14, MED15, MED16, MED17, MED18, MED19, MED20, MED21, MED22, MED23, MED24, MED25, MED26, MED27, MED28, MED29, MED30, and MED31. In some embodiments, the mediator complex subunit belongs to a head module and is selected from the group consisting of MED6, MED8, MED11, SRB4/MED17, SRB5/MED18, SRB2/MED20 and SRB6/MED22. In some embodiments, the mediator complex subunit is a backbone subunit, e.g., MED14. In some embodiments, the mediator complex subunit belongs to a middle module and is selected from the group consisting of MED1, MED4, NUT1/MED5, MED7, CSE2/MED9, NUT2/MED10, ROX3/MED19, SRB7/MED21, MED26, and SOH1/MED31. In some embodiments, the subunit of the middle module is MED19. In some embodiments, the subunit of the middle module is MED26. In some embodiments, the mediator complex subunit belongs to a tail module and is selected from the group consisting of MED2, PGD1/MED3, MED5, GAL11/MED15, SIN4/MED16, MED23, MED24, MED25, MED27, MED28, and MED30. In some embodiments, the subunit of the tail module is MED15. In some embodiments, the subunit of the tail module is MED16. In some embodiments, the subunit of the tail module is MED24. In some embodiments, the subunit of the tail module is MED27.

In some embodiments, the mediator complex subunit belongs to a CDK8 module (CKM) and is selected from the group consisting of MED12 (or MED12L), MED13 (or MED13L), CCNC, and CDK8 (or CDK19). In some embodiments, the subunit of the CKM is MED12. In some embodiments, the subunit of the CKM is MED13. In some embodiments, the subunit of the CKM is CCNC. In some embodiments, the subunit of the CDK8 module is CDK8. In some embodiments, the subunit of the CDK8 module is CDK19.

In some embodiments, the methods disclosed herein includes introducing into the immune cell a nucleic acid and/or a polypeptide capable of reducing (e.g., alleviating) expression level of one or more mediator complex subunits in the immune cell, wherein the one or more mediator complex subunit is selected from the middle module subunits, the tail module subunits, and the cyclin-dependent-kinase 8 (CDK8) module subunits of the mediator complex. In some embodiments, the mediator complex subunit is selected from the group consisting of CCNC, CDK8, CDK19, MED12, MED12L, MED13, MED13L, MED19, MED24, and MED26.

In some embodiments, the mediator complex subunit is a middle module subunit. In some embodiments, the middle module subunit is MED19 or MED26. In some embodiments, the mediator complex subunit is a tail module subunit. In some embodiments, the tail module subunit is MED15, MED16, or MED24. In some embodiments, the mediator complex subunit is a CDK8 module subunit. In some embodiments, the CDK8 module subunit is selected from the group consisting of CCNC, CDK18, CDK19, MED12, MED12L, and MED13. In some embodiments, the CDK8 module subunit is CCNC. In some embodiments, the CDK8 module subunit is MED12.

In some embodiments, the methods described herein include introducing into the immune cell a nucleic acid and/or a polypeptide capable of reducing expression level of one or more mediator complex subunits in the immune cell. In some embodiments, the nucleic acid includes a polynucleotide sequence having sufficient sequence complementarity to a target sequence within an endogenous genomic locus encoding the mediator complex subunit. In some embodiments, the polynucleotide sequence has sufficient sequence complementarity to a target sequence within an endogenous genomic locus encoding the mediator complex subunit to allow hybridization of the polynucleotide sequence to the target sequence within an endogenous genomic locus encoding the mediator complex subunit. In some embodiments, the polynucleotide sequence has at least 70%, at least 80%, at least 90%, at least 95%, at least 96%, at least 97%, at least 98%, or at least 99% sequence identity to its target sequence within an endogenous genomic locus encoding the mediator complex subunit. In some embodiments, the polynucleotide sequence has 100% sequence identity to the target sequence within an endogenous genomic locus encoding the mediator complex subunit except for one, two, three, four, or five mismatches. In some embodiments, the target sequence is within the promoter region of the endogenous genomic locus, e.g., within 1-kb upstream of the transcription start site. In some embodiments, the target sequence is within the coding region of the endogenous genomic locus.

For example, in some embodiments, the nucleic acid is incorporated into a genome-targeting nucleic acid that can direct the activities of an associated polypeptide (e.g., a site-directed endonuclease or DNA endonuclease) to a specific target sequence within a target nucleic acid. In some embodiments, the genome-targeting nucleic acid is an RNA. In some embodiments, a genome-targeting RNA is a “guide RNA” or “gRNA” herein. Generally, a guide RNA has at least a spacer sequence that hybridizes to a target nucleic acid sequence of interest and a CRISPR repeat sequence. For example, in Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed endonuclease such that the guide RNA and site-direct endonuclease form a complex. The genome-targeting nucleic acid provides target specificity to the complex by virtue of its association with the site-directed endonuclease. The genome-targeting nucleic acid thus directs the activity of the site-directed endonuclease. As described in greater detail below, CRISPR endonucleases, such as Cas9, can be used in various embodiments of the methods of the disclosure. Other suitable forms of endonucleases include, but are not limited to, zinc-finger nucleases (ZFNs), transcription activator-like effector nucleases (TALENs), homing endonucleases (HEs,) or MegaTALs, or combinations of nucleases.

In some embodiments, the genome-targeting nucleic acid is a double-molecule guide RNA. In some embodiments, the genome-targeting nucleic acid is a single-molecule guide RNA (sgRNA). A double-molecule guide RNA (dgRNA) has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence.

By way of illustration, guide RNAs used in the CRISPR/Cas/Cpf1 system, or other smaller RNAs can be readily synthesized by chemical means as illustrated below and described in the art. While chemical synthetic procedures are continually expanding, purifications of such RNAs by procedures such as high performance liquid chromatography (HPLC), which avoids the use of gels such as PAGE) tends to become more challenging as polynucleotide lengths increase significantly beyond a hundred or so nucleotides.

Accordingly, in some embodiments, the nucleic acid is incorporated into a guide RNA (gRNA) of a CRISPR/Cas genome editing system that can induce introduction of one or more molecular alterations (e.g., mutations, deletions, insertions, In/Del, substitutions) in the endogenous locus encoding the mediator complex subunit.

In some other embodiments, the nucleic acid is incorporated into a TALEN (transcription activator-like effector nuclease) genome editing system that can introduce one or more molecular alterations in the endogenous locus encoding the mediator complex subunit in the immune cell. In some other embodiments, the nucleic acid is incorporated into a DNA-guided endonuclease genome editing with NgAgo (Natronobacterium gregoryi Argonaute) in the immune cell. In some other embodiments, the nucleic acid is incorporated into an anti-sense nucleic acid molecule capable of inducing expression suppression of the endogenous locus encoding the mediator complex subunit in the immune cell. In some other embodiments, the nucleic acid is incorporated into a double-stranded RNAi molecule capable of causing expression suppression of the endogenous locus encoding the mediator complex subunit in the immune cell. In some other embodiments, the nucleic acid is incorporated into a single stranded RNA molecule capable of informing a hairpin structure and capable of inducing suppression or degradation of mRNA.

The basic techniques for operably linking two or more sequences of DNA together are familiar to the skilled worker, and such methods have been described in a number of texts for standard molecular biological manipulation. The molecular techniques and methods by which these nucleic acid molecules can be constructed and characterized are described more fully below and in the Examples herein.

In some embodiments of the disclosure, the nucleic acid is operably linked to a heterologous nucleic acid sequence. In some embodiments, the heterologous nucleic acid sequence includes a transcription control element or a coding sequence for a selectable marker. In some embodiments, the polynucleotide sequence with sufficient sequence complementary to an endogenous locus for a mediator complex subunit is operably linked to a transcription control element. In some embodiments, the transcription control element is a promoter sequence. A non-limiting exemplification of suitable promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, MoMuLV promoter, an avian leukemia virus promoter, an Epstein-Barr virus immediate early promoter, mouse mammary tumor virus (MMTV), human immunodeficiency virus (HIV) long terminal repeat (LTR) promoter, a Rous sarcoma virus promoter, the elongation factor-1a promoter, as well as human gene promoters such as, but not limited to, the actin promoter, the myosin promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the nucleic acid with sufficient sequence complementarity to a target sequence within an endogenous locus encoding the mediator complex subunit can be incorporated into an expression cassette or an expression vector. It will be understood that an expression cassette generally includes a construct of genetic material that contains coding sequences and enough regulatory information to direct proper transcription and/or translation of the coding sequences in a cell, in vivo and/or ex vivo. Generally, the expression cassette can be inserted into a vector for targeting to a desired host cell and/or into a desired host cell and/or into an individual. As such, in some embodiments, an expression cassette of the disclosure includes a polynucleotide sequence with sufficient sequence complementary to an endogenous locus for a mediator complex subunit as disclosed herein, which is operably linked to expression control elements, such as a promoter, and optionally, any or a combination of other nucleic acid sequences that affect the transcription or translation of the coding sequence. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules including a polynucleotide sequence with sufficient sequence complementary to an endogenous locus for a mediator complex subunit as described herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic cell, such as a mammalian cell (e.g., COS cells, NIH 3T3 cells, or HeLa cells). These cells are available from many sources, including the American Type Culture Collection (Manassas, VA). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009). Accordingly, the nucleic acid sequence including a polynucleotide sequence with sufficient sequence complementary to an endogenous locus for a mediator complex subunit can be incorporated into a viral vector. In some embodiments, the vector is a viral vector derived from a lentivirus, an adeno-virus, an adeno-associated virus, a baculovirus, or a retrovirus. In some embodiments, the nucleic acid is incorporated into a nucleic construct for use in guide RNA-directed CRISPR-mediated knock-in procedure, CRISPR/Cas9 genome editing, or DNA-guided endonuclease genome editing with NgAgo (Natronobacterium gregoryi Argonaute), or TALENs genome editing (transcription activator-like effector nucleases).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

In some embodiments, the immune cell is T lymphocyte, a natural killer (NK) cell, a natural killer T cell (NKT), or a macrophage. In some particular embodiments, the immune cell is T lymphocyte. In some embodiments, the T lymphocyte is a CD8+T cytotoxic lymphocyte cell selected from the group consisting of naïve CD8+ T cells, central memory CD8+ T cells, effector memory CD8+ T cells, effector CD8+ T cells, CD8+ stem memory T cells, bulk CD8+ T cells. In some embodiments, the lymphocyte is a CD4+T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells. In some embodiments, the immune cell is ex vivo. In some embodiments, the immune cell is in vitro. In some embodiments, the immune cell is in vivo. In some embodiments, the immune cell is an animal cell. In some embodiments, the animal cell is a mammalian cell. In some embodiments, the animal cell is a mouse cell. In some embodiments, the animal cell is a human cell. In some embodiments, the cell is a non-human primate cell. In some embodiments, the immune cell is obtained by leukapheresis performed on a sample obtained from a subject.

In some embodiments, the methods of the disclosure further include introducing into the immune cells one or more recombinant immune receptors, such as, such as a chimeric antigen receptor (CAR) or a T cell receptor (TCR), and/or nucleic acids encoding the same. For example, the immune cells can include and/or express an antigen-specific receptor, e.g., a receptor that can immunologically recognize and/or specifically bind to an antigen, or an epitope thereof, such that binding of the antigen-specific receptor to antigen, or the epitope thereof, elicits an immune response. In some embodiments, the antigen-specific receptor has antigenic specificity for a cancer antigen, such as a tumor-specific antigen (TSA) or a tumor-associated antigen (TAA).

In some embodiments, the antigen-specific receptor is a T-cell receptor (TCR). A TCR generally includes two polypeptides (e.g., polypeptide chains), such as an α-chain of a TCR, a β-chain of a TCR, a γ-chain of a TCR, a δ-chain of a TCR, or a combination thereof. Such polypeptide chains of TCRs are known in the art. The antigen-specific TCR can include any amino acid sequence, provided that the TCR can specifically bind to and/or immunologically recognize an antigen, such as a cancer antigen or epitope thereof. In some embodiments, the TCR is an endogenous TCR, e.g., a TCR that is endogenous or native to (naturally-occurring) the T cell. In such a case, the T cell expressing the endogenous TCR can be a T cell that was isolated from a mammal which is known to express the particular cancer antigen. For example, in some embodiments, the T cell is a primary T cell isolated from a mammal having a cancer. In some embodiments, the T cell is a TIL or a T cell isolated from a human cancer patient.

In some embodiments, the immune cells include and/or express a chimeric antigen receptor (CAR). Generally, a CAR includes an antigen binding domain, e.g., a single-chain variable fragment (scFv) of an antibody, fused to a transmembrane domain and an intracellular domain. In this case, the antigenic specificity of a CAR can be encoded by a scFv which specifically binds to the antigen, or an epitope thereof. CARs, and methods of making them, are known in the art.

In some embodiments, the immune cells include one or more nucleic acids encoding an exogenous (e.g., recombinant) antigen-specific receptor. In some embodiments, such exogenous antigen-specific receptors, e.g., exogenous TCRs and CARs can confer specificity for additional antigens to the T cell beyond the antigens for which the endogenous TCR is naturally specific.

In some embodiments, the reduced expression level of the one or more mediator complex subunits results in an improved function of CAR T cells, as indicated by for example increased production of interferon gamma (IFNγ), tumor-necrosis factor alpha (TNFα), and/or interleukin-2 (IL-2) relative to the production of these molecules in reference control cells, e.g., cells with native expression levels of mediator complex subunits. In some embodiments, the reduced expression of the one or more mediator complex subunits results in higher proliferative potential of CAR T cells. In some embodiments, the reduced expression of the one or more mediator complex subunits results in an enhanced effector function of the CAR T cells, such as, for example increased growth rate (proliferation), cytokine production, target cell inhibition (e.g., anti-cancer cytotoxicity), macrophage activation, T cell activation, NK cell activation, and in vivo persistence (e.g., survival). In some embodiments, the reduced expression of the one or more mediator complex subunits results in an increased effector memory T cell phenotype. In some embodiments, the reduced expression of the one or more mediator complex subunits results in increased oxygen consumption and extracellular acidification rate.

In one aspect, provided herein are immune cells that have been engineered to have a reduced (e.g., alleviated) level of one or more mediator complex subunits. In some embodiments, the mediator complex subunit is selected from the middle module subunits, the tail module subunits, and the cyclin-dependent-kinase 8 (CDK8) module subunits of the mediator complex. In some embodiments, the mediator complex subunit is selected from the group consisting of CCNC, CDK8, CDK19, MED12, MED12L, MED13, MED13L, MED19, MED24, and MED26. In some embodiments, the mediator complex subunit is a middle module subunit. In some embodiments, the middle module subunit is MED19 or MED26. In some embodiments, the mediator complex subunit is a tail module subunit. In some embodiments, the tail module subunit is MED15, MED16, or MED24. In some embodiments, the mediator complex subunit is a CDK8 module subunit. In some embodiments, the CDK8 module subunit is selected from the group consisting of CCNC, CDK18, CDK19, MED12, MED12L, and MED13. In some embodiments, the mediator complex subunit is CCNC. In some embodiments, the mediator complex subunit is MED12. Some embodiments of the disclosure provide engineered immune cells that have been produced by a method described herein. In some embodiments, the immune cells are in vitro. In some embodiments, the immune cells are ex vivo. In some embodiments, the immune cells are in vivo. In some embodiments, the immune cell is a T lymphocyte. In some embodiments, the immune cell is an exhausted immune cell or a non-exhausted immune cell. Accordingly, cell cultures including at least one engineered immune cell as disclosed herein and a culture medium are also within the scope of this application. Methods and systems suitable for generating and maintaining cell cultures are known in the art.

### Compositions of the Disclosure

The engineered immune cells and nucleic acids of the disclosure can be incorporated into compositions, including pharmaceutical compositions. Such compositions generally can include one or more engineered immune cells and nucleic acids of the disclosure. Accordingly, in one aspect, some embodiments of the disclosure relate to compositions including a) an engineered immune cell of the disclosure; and/or b) a nucleic acid including a sequence having sufficient sequence complementarity to a target sequence within a genomic locus encoding one or more mediator complex subunits. In some embodiments, the one or more mediator complex subunits is selected from the group consisting of CCNC, CDK8, CDK19, MED12, MED12L, MED13, MED13L, MED19, MED24, and MED26.

### Pharmaceutical Compositions

The engineered immune cells and nucleic acids of the disclosure can be incorporated into pharmaceutical compositions. Such compositions generally can include one or more engineered immune cells and nucleic acids of the disclosure and a pharmaceutically acceptable excipient, e.g., a carrier. Accordingly, in one aspect, some embodiments of the disclosure relate to pharmaceutical compositions including a pharmaceutically acceptable excipient and a) an engineered immune cell of the disclosure; and/or b) a nucleic acid including a sequence having sufficient sequence complementarity to a target sequence within a genomic locus encoding one or more mediator complex subunits. In some embodiments, the one or more mediator complex subunits is selected from the middle module subunits, the tail module subunits, and the cyclin-dependent-kinase 8 (CDK8) module subunits of the mediator complex. In some embodiments, the mediator complex subunit is selected from the group consisting of group consisting of CCNC, CDK8, CDK19, MED12, MED12L, MED13, MED13L, MED19, MED24, and MED26. In some embodiments, the mediator complex subunit is a middle module subunit. In some embodiments, the middle module subunit is MED19 or MED26. In some embodiments, the mediator complex subunit is a tail module subunit. In some embodiments, the tail module subunit is MED15, MED16, or MED24. In some embodiments, the mediator complex subunit is a CDK8 module subunit. In some embodiments, the CDK8 module subunit is selected from the group consisting of CCNC, CDK18, CDK19, MED12, MED12L, and MED13. In some embodiments, the mediator complex subunit is CCNC. In some embodiments, the mediator complex subunit is MED12.

Non-limiting exemplary embodiments of the pharmaceutical compositions described herein can include one or more of the following features. In some embodiments, the composition includes a nucleic acid molecule encoding one or more mediator complex subunits, and a pharmaceutically acceptable excipient. In some embodiments, the nucleic acid molecule is incorporated into an expression cassette or an expression vector. In some embodiments, the expression vector is a viral vector. In some embodiments, the viral vector is a lentiviral vector, an adenovirus vector, an adeno-associated virus vector, or a retroviral vector.

In some embodiments, the nucleic acid molecule can be introduced into a host immune cell, for example, a T lymphocyte, an NK cell, or a NKT cell, to produce a recombinant (e.g., engineered) immune cell containing the nucleic acid. In some embodiments, the nucleic acid molecule can be administered into a subject in need thereof.

Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecule can be delivered by viral or non-viral delivery vehicles known in the art. For example, the nucleic acid molecule can be stably integrated in the host genome, or can be episomally replicating, or present in the host cell as a mini-circle expression vector for transient expression. Accordingly, in some embodiments, the nucleic acid molecule is maintained and replicated in the host cell as an episomal unit. In some embodiments, the nucleic acid molecule is stably integrated into the genome of the host cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the host cell as a mini-circle expression vector for transient expression. In some embodiments, the nucleic acid molecule is incorporated into an anti-sense nucleic acid molecule that targets and suppresses expression of an endogenous genomic locus encoding a mediator complex subunit. In some embodiments, the nucleic acid molecule is incorporated into a double-stranded interference RNA (RNAi) molecule that targets and suppresses expression of an endogenous genomic locus encoding a mediator complex subunit. In some embodiments, the nucleic acid molecule is incorporated into an RNA molecule with a hairpin structure capable of targeting and degrading mRNAs encoding a mediator complex subunit.

The nucleic acid molecules can be encapsulated in a viral capsid, or a liposome, or a lipid nanoparticle (LNP), or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, the composition includes at least one engineered immune cell of the disclosure, and a pharmaceutically acceptable excipient. In some embodiments, the at least one engineered immune cell exhibits an enhanced effector function when introduced into a subject. Examples of effector functions that are enhanced in the engineered immune cells include, but are not limited to growth rate (proliferation), cytokine production, target cell inhibition (e.g., anti-cancer cytotoxicity), macrophage activation, T cell activation, NK cell activation, and in vivo persistence (e.g., survival). In some embodiments, the at least one engineered immune cell has an increased production of interferon gamma (INFY), interleukin-2 (IL-2), and/or tumor-necrosis factor α (TNFα).

In certain embodiments, the pharmaceutical compositions in accordance with some embodiments disclosed herein include cultures of engineered immune cells that can be washed, treated, combined, supplemented, or otherwise altered prior to administration to an individual in need thereof. Furthermore, administration can be at varied doses, time intervals or in multiple administrations.

The pharmaceutical compositions provided herein can be in any form that allows for the composition to be administered to a subject. In some specific embodiments, the pharmaceutical compositions are suitable for human administration. As used herein, the term “pharmaceutically acceptable” means approved by a regulatory agency of the Federal or a state government or listed in the U.S. Pharmacopeia or other generally recognized pharmacopeia for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual. In some embodiments, the individual is a human. One of ordinary skilled in the art will appreciate that the formulation should suit the mode of administration.

In some embodiments, the pharmaceutical compositions of the present disclosure are formulated to be suitable for the intended route of administration to an individual. For example, the pharmaceutical composition may be formulated to be suitable for parenteral, intraperitoneal, colorectal, intraperitoneal, and intratumoral administration. In some embodiments, the pharmaceutical composition may be formulated for intravenous, oral, intraperitoneal, intratracheal, subcutaneous, intramuscular, topical, or intratumoral administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the engineered immune cells of the disclosure can be formulated for administration to a subject using techniques known to the skilled artisan. For example, formulations comprising populations of engineered immune cells can include pharmaceutically acceptable excipient(s). Excipients included in the formulations will have different purposes depending, for example, on the engineered immune cells used and the mode of administration. Examples of generally used excipients included, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents. The formulations comprising engineered immune cells can have been prepared and cultured in the absence of non-human components, e.g., in the absence of animal serum. A formulation can include one population of engineered immune cells, or more than one, such as two, three, four, five, six or more populations of engineered immune cells.

Formulations comprising population(s) of engineered immune cells can be administered to a subject using modes and techniques known to the skilled artisan. Exemplary modes include, but are not limited to, intravenous injection. Other modes include, without limitation, intratumoral, intradermal, subcutaneous (S.C., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Devices useful for parenteral injection of infusion of the formulations can be used to effect such administration.

### Methods of Treatment

Administration of any one of the therapeutic compositions described herein, e.g., engineered immune cells, nucleic acids, and pharmaceutical compositions, can be used to treat patients in the treatment of relevant health conditions, such as proliferative diseases (e.g., cancers), autoimmune diseases, and microbial infections (e.g., viral infections). In some embodiments, one or more engineered immune cells, nucleic acids, and pharmaceutical compositions as described herein can be incorporated into therapeutic agents for use in methods of treating a subject who has, who is suspected of having, or who may be at high risk for developing one or more health conditions, such as proliferative diseases (e.g., cancers), autoimmune diseases, and chronic infections. In some embodiments, the individual is a patient under the care of a physician.

Accordingly, in one aspect, some embodiments of the disclosure relate to methods for preventing and/or treating a health condition in a subject in need thereof. In some embodiments, the methods include administering to the subject a composition of the disclosure. In some embodiments, the methods include administering to the subject a composition that includes an engineered immune cell of the disclosure. In some embodiments, the methods include administering to the subject a composition that includes a nucleic acid including a sequence having sufficient sequence complementarity to a target sequence within a genomic locus encoding a mediator complex subunit. In some embodiments, the nucleic acid includes a sequence having sufficient sequence complementarity to a target sequence within a genomic locus encoding a mediator complex subunits selected from the middle module subunits, the tail module subunits, and the cyclin-dependent-kinase 8 (CDK8) module subunits of the mediator complex. In some embodiments, the mediator complex subunit is selected from the group consisting of CCNC, CDK8, CDK19, MED12, MED12L, MED13, MED13L, MED19, MED24, and MED26. In some embodiments, the mediator complex subunit is a middle module subunit. In some embodiments, the middle module subunit is MED19 or MED26. In some embodiments, the mediator complex subunit is a tail module subunit. In some embodiments, the tail module subunit is MED15, MED16, or MED24. In some embodiments, the mediator complex subunit is a CDK8 module subunit. In some embodiments, the CDK8 module subunit is selected from the group consisting of CCNC, CDK18, CDK19, MED12, MED12L, and MED13. In some embodiments, the mediator complex subunit is CCNC. In some embodiments, the mediator complex subunit is MED12. In some embodiments, the methods include administering to the subject a pharmaceutical composition as described herein.

In some embodiments, the methods include administering a therapeutically effective amount of a composition of the disclosure (e.g., engineered immune cells, nucleic acid molecules, and pharmaceutical compositions) to a subject in need thereof. The term “effective amount”, “therapeutically effective amount”, or “pharmaceutically effective amount” of a subject engineered immune cell or pharmaceutical composition of the disclosure generally refers to an amount or number sufficient for a population of engineered immune cells or a pharmaceutical composition to accomplish a stated purpose relative to the absence of the engineered immune cell population or pharmaceutical composition (e.g., achieve the effect for which it is administered, treat a disease, reduce a signaling pathway, or reduce one or more symptoms of a disease or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a T-cell population or composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

Non-limiting exemplary embodiments of the treatment methods described herein can include one or more of the following features. In some embodiments, the health condition is a proliferative disease or an infection. Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma. In some embodiments, the cancer is leukemia.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

Exemplary proliferative diseases can include, without limitation, angiogenic diseases, a metastatic diseases, tumorigenic diseases, neoplastic diseases and cancers. In some embodiments, the proliferative disease is a cancer. The term “cancer” generally refers to a disease characterized by the rapid and uncontrolled growth of aberrant cells. The aberrant cells may form solid tumors or constitute a hematological malignancy. Cancer cells can spread locally or through the bloodstream and lymphatic system to other parts of the body. There are no specific limitations with respect to the cancers which can be treated by the compositions and methods of the present disclosure. Non-limiting examples of suitable cancers include ovarian cancer, renal cancer, breast cancer, prostate cancer, liver cancer, brain cancer, lymphoma, leukemia, cervical cancer, skin cancer, pancreatic cancer, colorectal cancer, lung cancer and the like.

Other cancers that can be suitable treated with the compositions and methods of the present disclosure include, but are not limited to, acute myeloblastic leukemia (AML), acute lymphoblastic leukemia (ALL), chronic myelocytic leukemia (CML), adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, colon and rectum cancer, endometrial cancer, esophagus cancer, Ewing's family of tumors (e.g. Ewing's sarcoma), eye cancer, transitional cell carcinoma, vaginal cancer, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, gallbladder cancer, gastrointestinal carcinoid tumors, gastrointestinal stromal tumors, gestational trophoblastic disease, Non-Hodgkin's lymphoma, Hodgkin's lymphoma, childhood Non-Hodgkin's lymphoma, Kaposi's sarcoma, kidney cancer, laryngeal and hypopharyngeal cancer, liver cancer, lung cancer, lung carcinoid tumors, malignant mesothelioma, multiple myeloma, myelodysplastic syndrome, rhabdomyosarcoma, salivary gland cancer, sarcomas, melanoma skin cancer, non-melanoma skin cancers, stomach cancer, testicular cancer, thymus cancer, thyroid cancer, uterine cancer (e.g., uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia. In some embodiments, the cancer is osteosarcoma.

Particularly suitable cancers include, but are not limited to, breast cancer, ovarian cancer, lung cancer, pancreatic cancer, mesothelioma, leukemia, lymphoma, brain cancer, prostate cancer, multiple myeloma, melanoma, bladder cancer, bone sarcomas, soft tissue sarcomas, retinoblastoma, renal tumors, neuroblastoma, and carcinomas.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. For example, in some embodiments, the composition administered to the subject can reduce metastatic nodules in the subject. In some embodiments, the administered composition inhibits tumor growth in the subject.

In some embodiments, the proliferative disease is an autoimmune disease. In some embodiments, the autoimmune disease is selected from the group consisting of rheumatoid arthritis, insulin-dependent diabetes mellitus, hemolytic anemias, rheumatic fever, thyroiditis, Crohn's disease, myasthenia gravis, glomerulonephritis, autoimmune hepatitis, multiple sclerosis, alopecia areata, psoriasis, vitiligo, dystrophic epidermolysis bullosa, systemic lupus erythematosus, moderate to severe plaque psoriasis, psoriatic arthritis, Crohn's disease, ulcerative colitis, and graft vs. host disease.

In some embodiments, the administered composition inhibits proliferation of a target cancer cell, and/or inhibits tumor growth of the cancer in the subject. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the engineered immune cells disclosed herein, wherein the engineered immune cells inhibit the proliferation of the target cell and/or inhibit tumor growth of a target cancer in the subject compared to the proliferation of the target cell and/or tumor growth of the target cancer in subjects who have not been administered with the engineered immune cells.

Administration of the compositions described herein, e.g., engineered immune cells, nucleic acids, and pharmaceutical compositions, can be used in the stimulation of an immune response. In some embodiments, one or more of engineered immune cells, nucleic acids, and/or pharmaceutical compositions as described herein are administered to an individual after induction of remission of cancer with chemotherapy, or after autologous or allogeneic hematopoietic stem cell transplantation. In some embodiments, compositions described herein are administered to a subject in need of increasing the production of interferon gamma (IFNγ), tumor-necrosis factor alpha (TNFα), and/or interleukin-2 (IL-2) in the treated subject relative to the production of these molecules in subjects who have not been administered one of the therapeutic compositions disclosed herein.

In some embodiments, the administered composition confers an enhanced effector function of the immune cells. Examples of effector functions of immune cell that can be enhanced in the engineered immune cells include, but are not limited to growth rate (proliferation), death rate, death rate type, target cell inhibition (cytotoxicity), target cell killing, target cell survival, cluster of differentiation change, macrophage activation, B cell activation, cytokine production, in vivo persistence. In some embodiments, the administered composition confers an increased effector memory T cell phenotype. In some embodiments, the administered composition confers an increased oxygen consumption and extracellular acidification rate. In some embodiments, an effector function of the immune cells including the composition of the disclosure is enhanced at levels that are at least 10% higher, such as at least 10% higher than about 10%, at least higher than about 20%, at least higher than about 30%, at least higher than about 40%, at least higher than about 50%, at least higher than about 60%, at least higher than about 70%, at least higher than about 80%, at least higher than about 90%, at least higher than about 2 times, higher than about three times, higher than about four time, higher than about five times, higher than about six times, higher than about seven times, higher than about eight times, higher than about nine times, higher than about 20 times, higher than about 50 times, higher than about 100 times, or higher than about 200 times compared to a reference immune cell. In some embodiments, the reference immune cell does not include a composition of the disclosure. In some embodiments, the administered composition confers an increased glycolytic flux in the immune cells. In some embodiments, the administered composition confers a glycolytic flux that is increased by at least 10%, such as at least about 10%, at least about 20%, at least about 30%, at least about 40%, at least about 50%, at least about 60%, at least about 70%, at least about 80%, at least about 90%, at least about 2 times, about three times, about four time, about five times, about six times, about seven times, about eight times, about nine times, about 20 times, about 50 times, about 100 times, or about 200 times compared to a reference immune cell (e.g., a non-engineered immune cell or untransduced immune cell).

An effective amount of the compositions described herein, e.g., engineered immune cells, nucleic acids, and/or pharmaceutical compositions, can be determined based on the intended goal, for example cancer regression. For example, where existing cancer is being treated, the amount of a composition disclosed herein to be administered may be greater than where administration of the composition is for prevention of cancer. One of ordinary skill in the art would be able to determine the amount of a composition to be administered and the frequency of administration in view of this disclosure. The quantity to be administered, both according to number of treatments and dose, also depends on the individual to be treated, the state of the individual, and the protection desired. Precise amounts of the composition also depend on the judgment of the practitioner and are peculiar to each subject. Frequency of administration could range from 1-2 days, to 2-6 hours, to 6-10 hours, to 1-2 weeks or longer depending on the judgment of the practitioner.

Determination of the amount of compositions to be administered will be made by one of skill in the art, and will in part be dependent on the extent and severity of cancer, and whether the engineered immune cells are being administered for treatment of existing cancer or prevention of cancer. For example, longer intervals between administration and lower amounts of compositions may be employed where the goal is prevention. For instance, amounts of compositions administered per dose may be 50% of the dose administered in treatment of active disease, and administration may be at weekly intervals. One of ordinary skill in the art, in light of this disclosure, would be able to determine an effective amount of compositions and frequency of administration. This determination would, in part, be dependent on the particular clinical circumstances that are present (e.g., type of cancer, severity of cancer).

In some embodiments, it may be desirable to provide a continuous supply of a composition disclosed herein to the subject to be treated, e.g., a patient. In some embodiments, continuous perfusion of the region of interest (such as a tumor) may be suitable. The time period for perfusion would be selected by the clinician for the particular subject and situation, but times could range from about 1-2 hours, to 2-6 hours, to about 6-10 hours, to about 10-24 hours, to about 1-2 days, to about 1-2 weeks or longer. Generally, the dose of the composition via continuous perfusion will be equivalent to that given by single or multiple injections, adjusted for the period of time over which the doses are administered.

In some embodiments, administration is by intravenous infusion. An effective amount of the engineered immune cells, nucleic acids, and/or pharmaceutical compositions disclosed herein can be determined based on the intended goal, for example tumor regression. For example, where existing cancer is being treated, the number of cells to be administered may be greater than where administration of the engineered immune cells disclosed herein is for prevention of cancer. One of ordinary skill in the art would be able to determine the number of cells to be administered and the frequency of administration in view of this disclosure. The quantity to be administered, both according to number of treatments and dose, also depends on the individual to be treated, the state of the individual, and the protection desired. Precise amounts of the therapeutic composition also depend on the judgment of the practitioner and are peculiar to each individual. Frequency of administration could range from 1-2 days, to 2-6 hours, to 6-10 hours, to 1-2 weeks or longer depending on the judgment of the practitioner. Generally, the dose of the therapeutic composition via continuous perfusion will be equivalent to that given by single or multiple injections, adjusted for the period of time over which the doses are administered.

### Administration of Engineered Immune Cells to a Subject

In some embodiments, the methods of the disclosure involve administering an effective amount or number of the engineered immune cells provided here to a subject in need thereof. This administering step can be accomplished using any method of implantation delivery in the art. For example, the engineered immune cells can be infused directly in the subject's bloodstream or otherwise administered to the subject.

In some embodiments, the methods disclosed herein include administering, which term is used interchangeably with the terms “introducing,” implanting,” and “transplanting,” engineered immune cells into an individual, by a method or route that results in at least partial localization of the introduced cells at a desired site such that a desired effect(s) is/are produced. The engineered immune cells or their differentiated progeny can be administered by any appropriate route that results in delivery to a desired location in the individual where at least a portion of the administered cells or components of the cells remain viable. The period of viability of the cells after administration to a subject can be as short as a few hours, e.g., twenty-four hours, to a few days, to as long as several years, or even the lifetime of the individual, e.g., long-term engraftment.

When provided prophylactically, the engineered immune cells described herein can be administered to a subject in advance of any symptom of a disease or health condition to be treated. Accordingly, in some embodiments the prophylactic administration of an engineered immune cell population prevents the occurrence of symptoms of the disease or health condition.

When provided therapeutically in some embodiments, engineered immune cells are provided at (or after) the onset of a symptom or indication of a disease or health condition, e.g., upon the onset of disease or health condition.

For use in the various embodiments described herein, an effective amount of engineered immune cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof.

In some embodiments, the engineered immune cells are non-autologous to the subject in need of treatment. In some embodiments, the adoptive cell therapy is an allogeneic adoptive cell therapy. For example, in some embodiments, the engineered immune cells are allogeneic to the subject in need of treatment. In an allogeneic adoptive cell therapy, the engineered immune cells are not derived from the individual receiving the adoptive cell therapy. Allogeneic cell therapy generally refers to a therapy whereby the individual (donor) who provides the immune cells is a different individual (of the same species) than the individual receiving the cell therapy. For example, a population of engineered immune cells being administered to an individual is derived from one more unrelated donors, or from one or more non-identical siblings. Accordingly, the engineered immune cells can be derived from one or more donors or can be obtained from an autologous source. In some embodiments, the engineered immune cells are expanded in culture prior to administration to a subject in need thereof.

In some embodiments, the delivery of a cell composition (e.g., a composition including a plurality of engineered immune cells according to any of the cells described herein) into a subject by a method or route results in at least partial localization of the cell composition at a desired site. A composition including engineered immune cells can be administered by any appropriate route that results in effective treatment in the subject, e.g., administration results in delivery to a desired location in the subject where at least a portion of the composition delivered, e.g., at least 1×10⁴ cells, is delivered to the desired site for a period of time. Modes of administration include injection, infusion, instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is often considered a standard mode of administration.

In some embodiments, the engineered immune cells are administered systemically, e.g., via infusion or injection. For example, a population of engineered immune cells as described herein are administered other than directly into a target site, tissue, or organ, such that it enters, the subject's circulatory system and, thus, is subject to metabolism and other similar biological processes.

The efficacy of a treatment including any of the compositions provided herein for the prevention or treatment of a disease or health condition can be determined by a skilled clinician. However, one skilled in the art will appreciate that a prevention or treatment is considered effective if any one or all of the signs or symptoms or markers of disease are improved or ameliorated. Efficacy can also be measured by failure of a subject to worsen as assessed by decreased hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

Measurement of the degree of efficacy is based on parameters selected with regard to the disease being treated and the symptoms experienced. In general, a parameter is selected that is known or accepted as correlating with the degree or severity of the disease, such as a parameter accepted or used in the medical community. For example, in the treatment of a solid cancer, suitable parameters can include reduction in the number and/or size of metastases, number of months of progression-free survival, overall survival, stage or grade of the disease, the rate of disease progression, the reduction in diagnostic biomarkers (for example without limitation, a reduction in circulating tumor DNA or RNA, a reduction in circulating cell-free tumor DNA or RNA, and the like), and combinations thereof. It will be understood that the effective dose and the degree of efficacy will generally be determined with relation to a single subject and/or a group or population of subjects. Therapeutic methods of the disclosure reduce symptoms and/or disease severity and/or disease biomarkers by at least about 1, 2, 3, 4, 5, 10, 15, 20, 25, 30, 35, 40, 45, 50, 55, 60, 65, 70, 75, 80, 85, 90, 95, 96, 97, 98, 99, or 100%.

As discussed above, a therapeutically effective amount of a pharmaceutical composition can be an amount of the pharmaceutical composition that is sufficient to promote a particular beneficial effect when administered to a subject, such as one who has, is suspected of having, or is at risk for a disease or health condition. In some embodiments, an effective amount includes an amount sufficient to prevent or delay the development of a symptom of the disease or health condition, alter the course of a symptom of the disease or health condition (for example but not limited to, slow the progression of a symptom of the disease), or reverse a symptom of the disease or health condition. It is understood that for any given case, an appropriate effective amount can be determined by one of ordinary skill in the art using routine experimentation.

### Additional Therapies

As discussed above, any one of the compositions as disclosed herein, e.g., engineered immune cells and pharmaceutical compositions, can be administered to a subject in need thereof as a single therapy (e.g., monotherapy). In addition or alternatively, in some embodiments of the disclosure, one or more of the engineered immune cells and pharmaceutical compositions described herein can be administered to the subject in combination with one or more additional therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins such as irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of prevention and/or treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. Non-limiting examples of immune checkpoint molecules include CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. Examples of antagonistic antibodies suitable for the compositions and methods disclosed herein include, but are not limited to, ipilimumab, nivolumab, pembrolizumab, durvalumab, atezolizumab, tremelimumab, and avelumab.

In some aspects, the one or more anti-cancer therapy is radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of pre-cancers or normal tissues is also contemplated herein. Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are kits for the practice of a method described herein. A kit can include one or more of the engineered immune cells and pharmaceutical compositions as described and provided herein. For examples, provided herein, in some embodiments, are kits that include one or more engineered immune cells of the disclosure. In some embodiments, provided herein are kits that include one or more pharmaceutical compositions of the disclosure. In some embodiments, the kits of disclosure further include written instructions for making the engineered immune cells, nucleic acids, and pharmaceutical compositions of the disclosure and using the same.

In some embodiments, the kits of the disclosure further include one or more syringes (including pre-filled syringes) and/or catheters (including pre-filled syringes) used to administer one any of the provided immune cells, nucleic acids, and pharmaceutical compositions to a subject in need thereof. In some embodiments, a kit can have one or more additional therapeutic agents that can be administered simultaneously or sequentially with the other kit components for a desired purpose, e.g., for modulating an activity of a cell, inhibiting a target cancer cell, or treating a health condition in a subject in need thereof.

For example, any of the above-described kits can further include one or more additional reagents, where such additional reagents can be selected from: dilution buffers; reconstitution solutions, wash buffers, control reagents, control expression vectors, negative control T-cell populations, positive control T-cell populations, reagents for ex vivo production of the T-cell populations.

In some embodiments, the components of a kit can be in separate containers. In some other embodiments, the components of a kit can be combined in a single container.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

Each of the aspects and embodiments described herein are capable of being used together, unless excluded either explicitly or clearly from the context of the embodiment or aspect.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### General Experimental Procedures

### T-Cell Isolation

Whole blood buffy coats were obtained from the Stanford Blood Center from volunteers under the age of 41 years. T cells were isolated using the RosetteSep™ Human T Cell Enrichment Cocktail (StemCell Technologies). T cells were stored in CryoStor® cell cryopreservation media CS10 (Sigma Aldrich) in liquid nitrogen.

### CRISPR Screen

### T-Cell Activation and Culturing

200 million T cells were thawed on Day 0 and activated with CD3/CD28 Dynabeads (Invitrogen) at a ratio of three beads per T cell. Cells were cultured in AIM-V medium (Gibco) supplemented with 5% FBS, HEPES, Penicillin, Streptomycin, and 10 mg/L IL-2. Cells were maintained at a density between 0.5 and 1 million per mL in T175 flasks.

### Lentiviral Transduction

All 9 pools of the Bassik Human CRISPR Knockout Library were obtained from Addgene and amplified with Endura ElectroCompetent Cells (Lucigen). LentiX cells (Takara) were plated on 150 mm plates coated with poly-D-lysine (Corning) and transfected with 18 μg REV, 18 μg GAG/POL, 7 μg VSVg, 15 μg library vector, 3.38 mL Opti-MEM (Gibco) and 135 μL Lipofectamine 2000 (Invitrogen) per plate. 10 plates were prepared for each donor. Media was changed 24 hours after transfection and supernatant was harvested 48 hours after transfection. Lentiviral supernatant was concentrated with lentiX (Takara) and added to the T cell culture medium 2 days post activation. On Day 3, cells were assessed for mCherry positivity by flow to confirm transduction was between 8 and 12%.

### CAS9 Electroporation

On Day 3, 100 μl reactions were assembled with 10 million T cells, 30 μg Alt-R® S.p. Cas9 Nuclease V3 (IDT), 90 μl P3 buffer (Lonza), and 7 μL Duplex Buffer (IDT). Cell were pulsed with protocol EO115 using the P3 Primary Cell 4D-Nucleofector™ L Kit and 4D Nucleofector™ System (Lonza). Cells were recovered immediately with 1 mL warm media, then transferred to 200 mL warm media for 6 hours prior to transduction with CAR.

### Retroviral Transduction

293GP cells were plated on 150 mm plates coated with poly-D-lysine (Corning) and transfected with 11 μg RD114, 22 μg HA-28z CARencoding plasmid, 3.38 mL Opti-MEM (Gibco) and 135 μL Lipofectamine 2000 (Invitrogen) per plate. The HA-28z CAR used in this experiment encoded 14g2a-E101K scFv, which demonstrate higher affinity (HA) for GD2, a disialoganglioside expressed naturally on tumor cells (Lynn et al., Nature, 2019). Media was changed 24 hours after transfection and supernatant was harvested 48 hours and 72 hours after transfection. 10 plates were prepared for each donor. On Day 2 and 3, non-tissue culture treated 12 well plates (Falcon) were coated with RetroNectin (Takara) and incubated at 4° C. overnight. On Days 3 and 4, 12 well plates were blocked with 2% BSA for 5 minutes and incubated with 1 mL retroviral supernatant per well for 2 hours at 32° C., 3200 rpm. 780 μl of supernatant was removed and 1 million T cells were added to each well in 1 mL of media. On Day 5, Dynabeads were removed using magnetic separation. Cells were cultured with puromycin at 2.5 μg/mL from Days 7 to 10 to eliminate cells which did not express a guide.

### Proliferation Screen

The CAR-T cell library was cultured in T175 flasks with passaging every other day. On Day 15, 100 million NALM6-GD2 cells were added to 100 million T cells and co-cultured to Day 23. 50% of the culture volume was discarded at each passage.

### Cytokine Production Screen

On Day 15, 100 million CAR-T cells co-cultured for 6 hours with 100 million NALM6-GD2-GFP tumor cells with eBioscience Monensin Solution (Invitrogen) in 200 mL. Intracellular cytokine staining was performed using the BD Cytofix/Cytoperm™ Fixation/Permeabilization Solution Kit. Cells were stained with antibodies purchased from Biolegend specific for CD4 (clone SK3), CD8 (clone SK1), TNF-α (clone MAb11), and IL-2 (clone MQ1-17H12), and fixable viability dye eFluor 506 (eBioscience). Cell sorting was performed at the Stanford Shared FACS Facility on a FACSAria II equipped with a 70 μm nozzle. The top 10% of TNFα+ and IL-2+ were sorted using individual gates for CD4+ and CD8+ cells. CD4+ and CD8+ cytokine high cells were pooled for genomic DNA extraction.

### Genomic DNA Extraction and Sequencing Library Preparation

Technical duplicates were performed for genomic DNA extraction, library preparation, and sequencing. 20-30 million cells were collected at each timepoint, with the exception of the sorted condition in which only one sample was processed for each donor, and each sample had about 5 million cells. For Day 0, the maxiprep of the lentiviral vector plasmid library was used as a proxy to approximate the relative abundance of guides in T cells at the start of the experiment, and duplicate sequencing libraries were prepared from maxiprep DNA were Genomic DNA was extracted from cell pellets using overnight lysis in SDS with proteinase K at 37° C. as previously described. Briefly, protein was precipitated with ammonium acetate and genomic DNA was precipitated with isopropanol. All of the recovered genomic DNA was used as template for PCR to generate the sequencing libraries. The libraries were prepared as described by Morgens et al. 2017. Illumina adapters were added using custom primers and deep sequencing was performed on the Illumina NovaSeq 6000 PE150 platform. Sequencing was performed by Novogene (Sacramento, CA).

### CRISPR Screen Data Analysis

BCL files were converted to FASTQ files using bcl2fastq2 Conversion Software v2.20. Guide sequences were extracted from FASTQ files and matched to the Bassik library index using a custom R script. Raw counts for each guide were provided as input to the MAGECK algorithm (Li 2014). For the proliferation screen, two replicates from “Day 0” were compared to 4 samples collected on Day 23 (two from each donor). For the cytokine production screen, 4 samples collected on Day 15 (two from each donor) were compared to 2 samples (1 from each donor) that were sorted for high cytokine expression. The MAGECK algorithm performs normalization, calculates log fold changes for guides and genes, and calculates adjusted p values.

TABLE 1
Properties of chimeric antigen receptors described herein.

| CAR | Target | Co-stimulation | Tonic signaling |
|---|---|---|---|
| CD19-28ζ | CD19 | CD28 | low |
| HA-28ζ | GD2 | CD28 | high |
| HER2-4-1BBζ | HER2 | 4-1BB | intermediate |

TABLE 2
Properties of synthetic single guide
RNAs used in this study. Analysis
of gene deletion efficiency was
performed with Synthego ICE
Analysis tool (v2).

| | | | | Indel | | Deletion |
|---|---|---|---|---|---|---|
| sgRNA | Target | Sequence | Source | % | R² | % |
| AAVS1-1 | AAVS1 | GGGGCCACT | Rose JC | 80 | 0.83 | 61 |
| | | AGGGACAGGAT | et al., | | | |
| | | (SEQ ID NO: 1) | 2017 | | | |
| sgKM040 | CCNC | GATGCCAAAAA | Morgens | 93 | 0.94 | 93 |
| | | CACACATGT | DW et | | | |
| | | (SEQ ID NO: 2) | al., 2017 | | | |
| sgKM041 | MED12 | GAACACCGGC | Morgens | nd | nd | nd |
| | | CCCTGAAG | DW et | | | |
| | | (SEQ ID NO: 3) | al., 2017 | | | |
| sgKM048 | MED12 | CCUGCCUCAG | This | 95 | 0.97 | 81 |
| | | GAUGAACUGA | study | | | |
| | | (SEQ ID NO: 4) | | | | |
| sgKM049 | MED12 | UAACCAGCCU | This | 97 | 0.95 | 93 |
| | | GCUGUCUCUG | study | | | |
| | | (SEQ ID NO: 5) | | | | |
| sgKM046 | CCNC | GGAUUUAAAG | This | 95 | .96 | 90 |
| | | UUUCUCUCAG | study | | | |
| | | (SEQ ID NO: 12) | | | | |

TABLE 3
Primers used to amplify genomic
DNA sequences for detection of
CRISPR/Cas9 editing.

| Primer | Sequence | Target |
|---|---|---|
| oKM.061 | CACCTAGGACGCACCATTCTCACA | AAVS1 |
| | (SEQ ID NO: 6) | forward |
| oKM.062 | CTTCTCCGACGGATGTCTCCCT | AAVS1 |
| | (SEQ ID NO: 7) | reverse |
| oKM.160 | GAGTGATGTTTGAGGGCGCG | MED12 |
| | (SEQ ID NO: 8) | forward |
| oKM.161 | CGACCAGTGTCAGGAAGGGTAT | MED12 |
| | (SEQ ID NO: 9) | reverse |
| oKM.139 | CAACAAGTTATTGCCACTGCTA | CCNC |
| | CGG (SEQ ID NO: 10) | forward |
| oKM.140 | ATCAGGATGATGGGAGGGAAGA | CCNC |
| | CAG (SEQ ID NO: 11) | reverse |

### Example 2

### Proliferation Screening of a CRISPR-Mediated Knock-Out CAR-T Cell Library

This Example describes the experimental design and results of a proliferation screening of CRISPR-mediated knock-out CAR-T cell library, where loss of a number of mediator subunits was found to result in reduced proliferation of CAR-T cells.

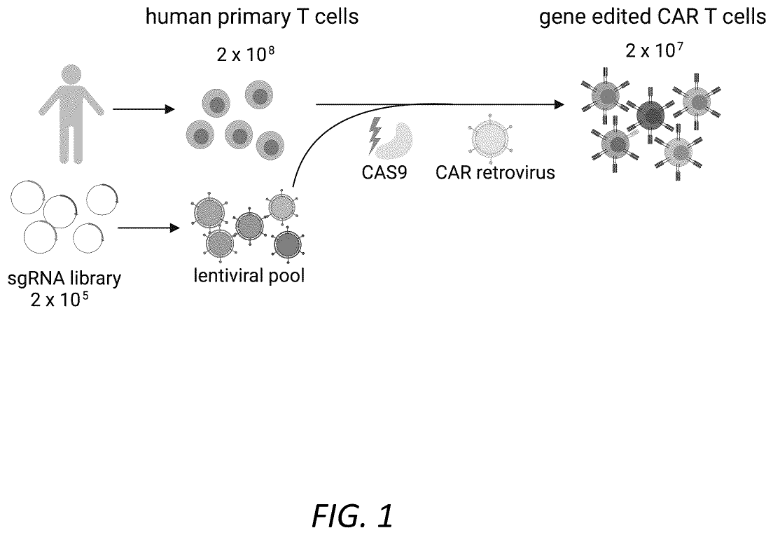

FIG. 1 schematically depicts the generation of CAR-T cell CRISPR knock out library used in these experiments. T cells were purified from two human donors. A CRISPR library targeting all ˜20,000 protein coding genes with 10 guides per gene was integrated into 200 million T cells at low multiplicity of infection (10% positive) using lentiviral vector. Purified Cas9 protein was electroporated into T cells on Day 3, and CAR was integrated by retrovirus on Days 3 and 4 post activation.

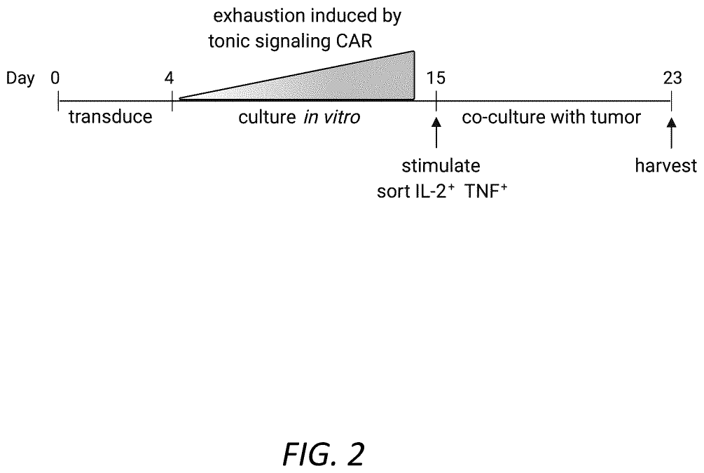

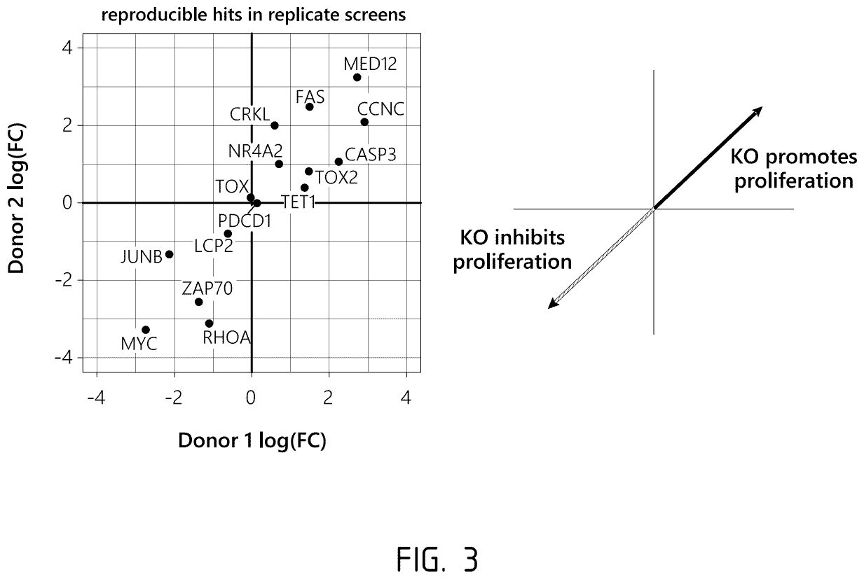

Experimental design of the CRISPR screen is depicted in FIG. 2. In this screen, gene edited CAR-T cells were cultured in vitro for 2 weeks, where expression of a tonic signaling CAR induced progressive T cell dysfunction. To screen for cytokine production, a fraction of the cultured population was stimulated with tumor and sorted by FACS for cells that expressed high levels of cytokine IL-2 and TNFα. To screen for proliferation, T cells were co-cultured with tumor cells expressing the CAR-T target for 7 more days. The screen result is presented in FIG. 3, where it was observed that CAR-T cells with knock-out mutations in genes selected from proliferation screen demonstrated reproducibility between replicate donors. In these experiments, the abundance of guide RNAs was quantified at Day 0 and at Day 23. The plot shows the average log 2 (fold change) of all guides targeting each gene. Genes plotted in the upper right quadrant were found to enhance proliferation when deleted (e.g., MED12 and CCNC), and genes plotted in the lower left quadrant were found decrease proliferation when deleted (e.g., MYC and RHOA).

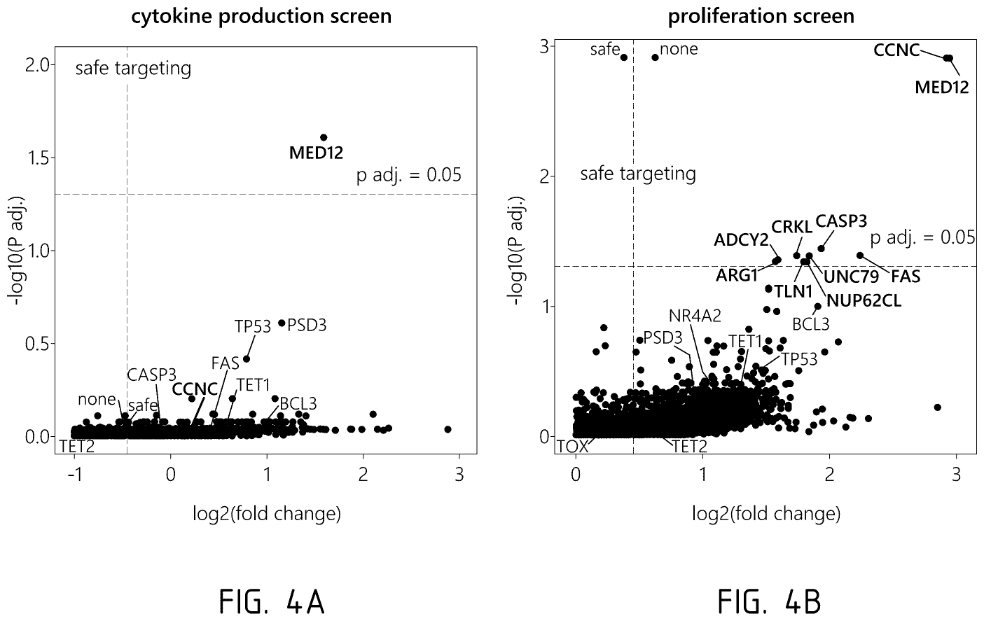

Additionally, a genome-wide CRISPR screen was also performed to identify statistically significant genes. In these experiments, the MAGECK algorithm was used to analyze the relative abundance of guides and calculate adjusted P values for each gene (Li 2014). As shown in FIG. 4A, the cytokine production screen compared guide abundance in the total Day 15 population to the cytokine high population. As shown in FIG. 4B, the proliferation screen compared abundance on Day 23 to Day 0. In these experiments, guides targeting safe, non-coding regions of the genome were included as controls and the average log 2 (fold change) for the safe targeting guides is indicated by the vertical dashed line. The threshold for statistical significance is indicated by the horizontal dashed line. The cytokine production screen identified 1 statistically significant genes, while the proliferation assay identified several statistically significant gene.

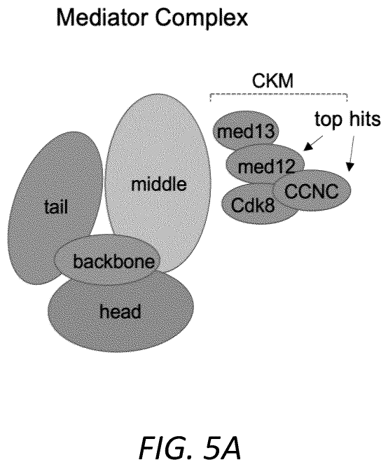

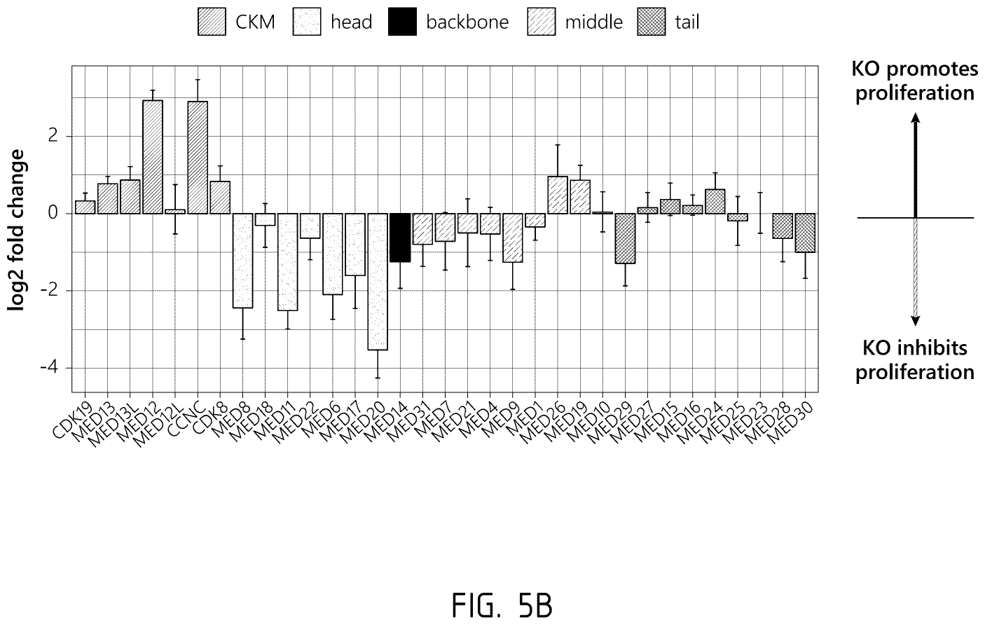

In particular, as shown in FIGS. 5A-5B, all 33 mediator complex subunits were detectable in the proliferation screen described above. In these figures, average log 2 (fold change) for all guides targeting each gene is plotted. First, an average enrichment for each guide was calculated by averaging donor 1 and donor 2. Then, the average enrichment for each gene was calculated by averaging the guide averages. Error bars depict standard deviation of the guides. MED12 and CCNC are both found in the CDK8 Kinase Module (CKM). It was observed that loss of all members of the CKD8 module (CKM) enhanced proliferation, with the exception of MED12L, which is not expressed in T cells. It was also observed that loss of mediator subunits found in the head module, backbone, and middle domains reduced proliferation, with the exception of MED26 and MED19. Without being bound to any particular theory, it was anticipated that MED26 and MED19 may form the physical contacts between the middle domain and the CKM.

Additionally, it was also observed that a few members of the tail region slightly enhanced proliferation when deleted (e.g., MED15, MED16, MED24, and MED27).

In particular, the CRISPR screening described herein indicate that reduction of MED12 expression in engineered T cells results in superior proliferation and cytokine production of the T cells. This result for MED12 was found to be consistent in two different human donors. Similar result was observed for CCNC, which strongly implicates the CKM as having a critical role in regulating T cell proliferation, since both MED12 and CCNC are subunits of the CKM. Furthermore, as described in greater detail below, examination of all mediator subunits indicates the essential role of the mediator complex in regulating T cell proliferation.

### Example 3

### Engineered CAR-T Cells Lacking MED12 or CCNC Subunit Produce More IL-2 and IFNγ

This Example describes the results of experiments performed to demonstrate that engineered CAR-T cells lacking MED12 or CCNC subunit (MED12-null and CCNC-null CAR-T cells) exhibit enhanced production of cytokines, exemplified by IL-2 and IFNγ.

In these experiments, target genes CCNC and MED12 and control gene AAVS1 were deleted on Day 3 post-activation. CAR-T cells were generated using the CD19-28ζ, HA-28ζ, and HER2-4-1BBζ receptors, cultured until Day 10 or 15, and subsequently co-cultured with NALM6, NALM6-GD2, or 143B cell lines, respectively. 24 hours after the addition of tumor cells, supernatants were collect and cytokines were quantified by ELISA. Mock-transduced T cells did not express a CAR and were included for a negative control. The bar graphs depict averages of two technical replicates, and error bars show the standard deviation.

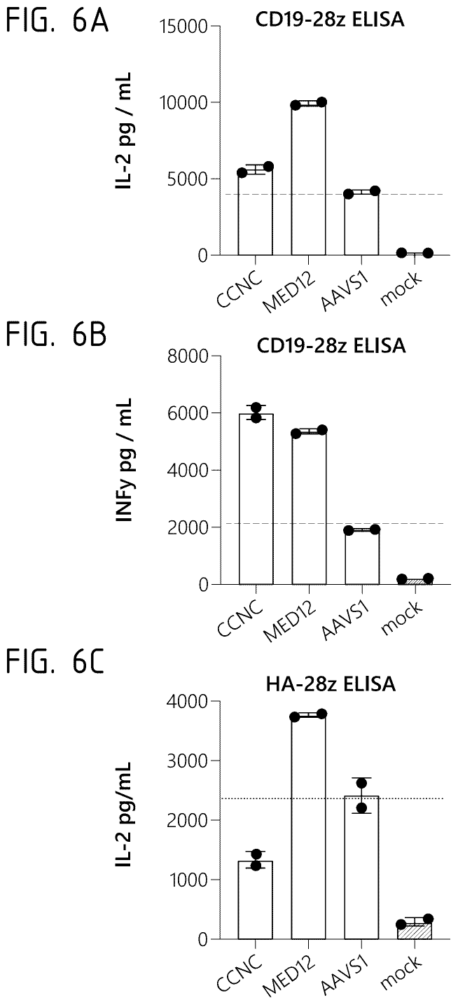

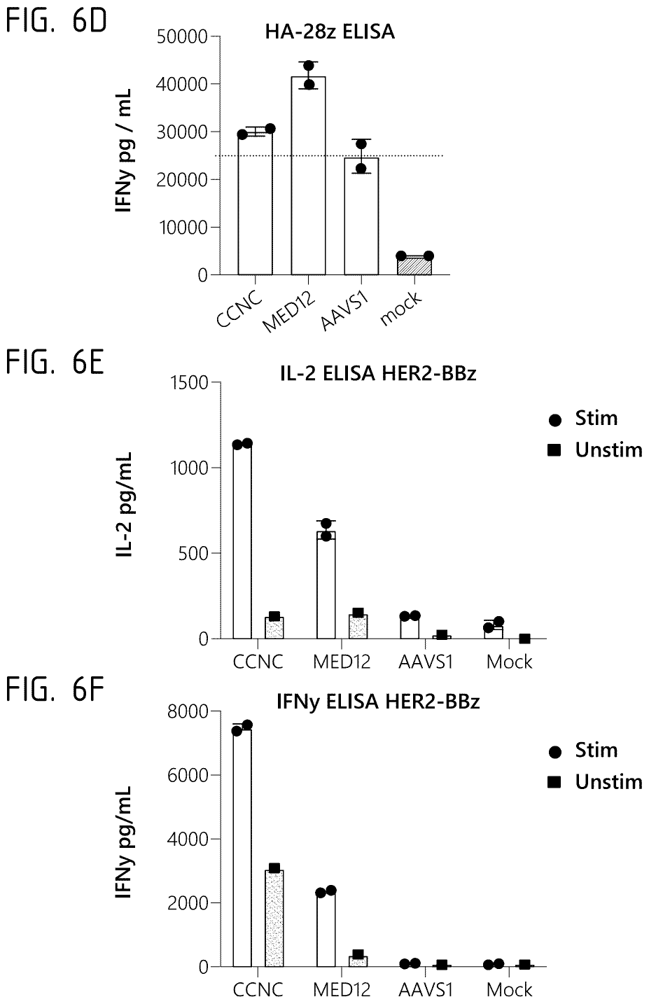

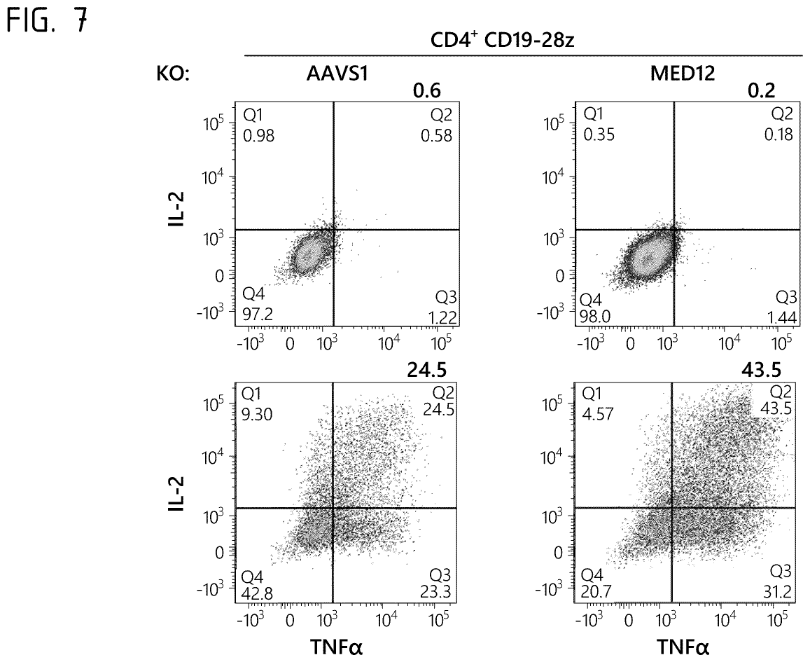

As shown in FIGS. 6A-6F, it was observed that MED12-null and CCNC-null CAR-T cells were capable of enhance production of IL-2 and IFNγ.

### Example 4

### MED12-Null CAR-T Cells Produce More IL-2 and TNFα on a Single-Cell Basis

This Example describes the results of experiments performed to illustrate that MED12-null CAR-T cells produce more IL-2 and TNFα on a single-cell basis.

In these experiments, CD19-28ζCAR-T cells were stimulated with NALM6 tumor cells on day 15 in the presence of monensin for 6 hours. Unstimulated (upper panel) and stimulated cell (lower panel) were fixed and stained for IL-2 and TNFα and analyzed by flow cytometry. The percentage of IL-2+ TNFα+ cells out of total CD4+ cells is displayed above each plot.

The experimental data presented in Examples 3 and 4 indicate loss-of-function mutations in the CKM module increases the number of T cells that have the capacity to secrete multiple pro-inflammatory cytokines. INFγ and TNFα have direct anti-tumor effects, while IL-2 promotes T cell proliferation. Without being bound to any particular theory, this increased capacity for cytokine secretion helps to explain why CCNC-null and MED12-null CAR-T cells have enhanced proliferation and increased tumor clearance.

### Example 5

### MED12-Null and CCNC-Null CD19-28ζCAR-T Cells Exhibit Enhanced Proliferation in Culture

This Example describes the results of experiments performed to illustrate that MED12-null and CCNC-null CD19-28ζ CAR-T cells proliferate more in culture.

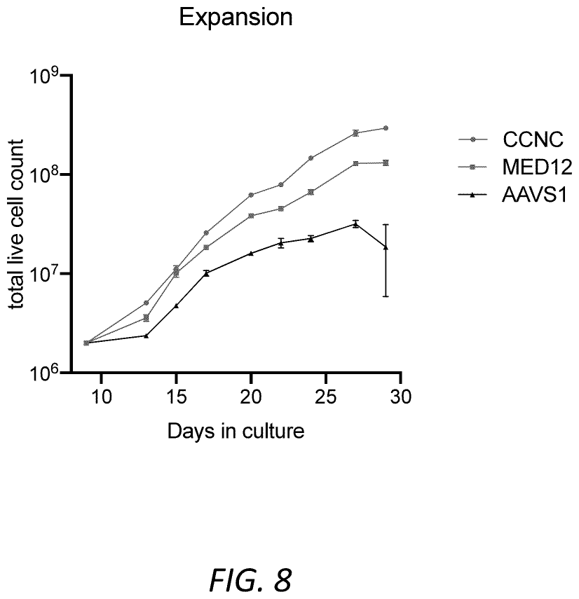

In these experiments, target genes CCNC and MED12 and control gene AAVS1 were deleted on Day 3 post-activation and cells were cultured with IL-2 until day 28. Average total live cells counts are plotted and error bars depict standard deviation of three technical replicates. As presented in FIG. 8, it was observed that MED12-null and CCNC-null CD19-28ζCAR-T cells proliferate more in culture.

### Example 6

### MED12-Null and CCNC-Null CD19-28ζCAR-T Cells are Dependent on IL-2 for Survival

This Example describes the results of experiments illustrating that MED12-null and CCNC-null CD19-28ζ CAR-T cells are dependent on IL-2 for survival.

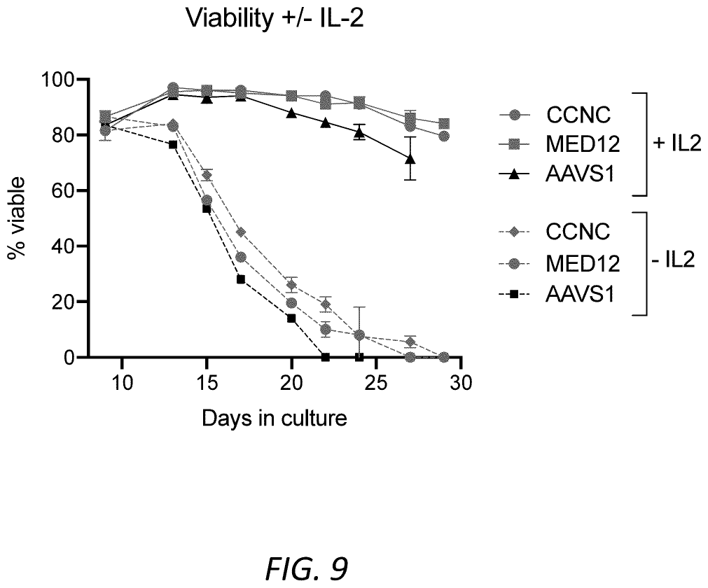

In these experiments, on Day 9, cells were washed and plated in media with or without IL-2. Cell density was maintained between 0.5 and 1 million cells per mL medium. Cells were stained with acridine orange and propidium iodide and viability was monitored with the CellacaMX cell counter. In the absence of IL-2, no viable cells were detected in any condition by Day 28. As shown in FIG. 9, it was observed that MED12-null and CCNC-null CD19-28ζ CAR-T cells are dependent on IL-2 for survival.

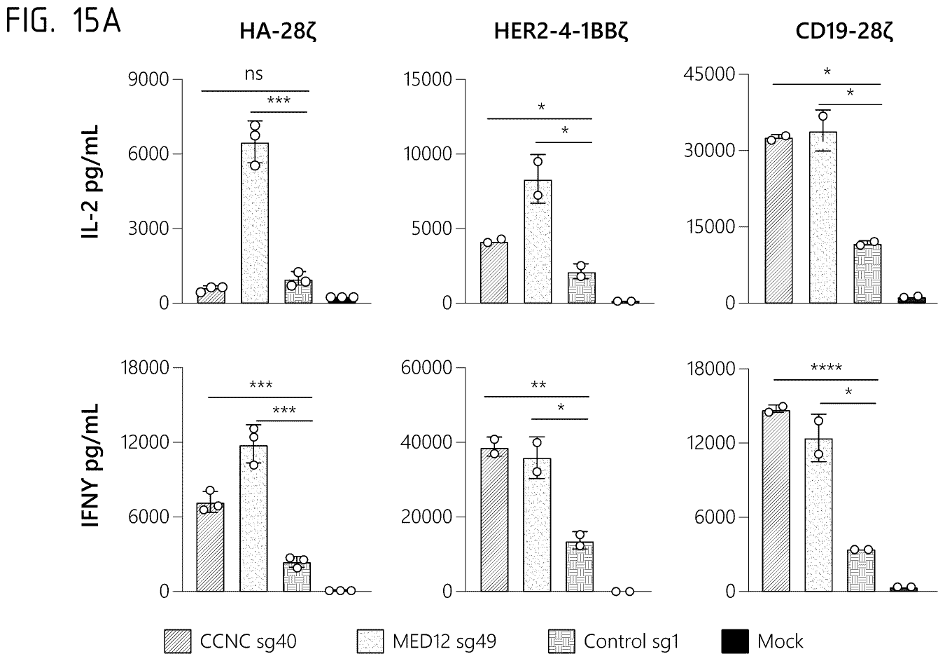

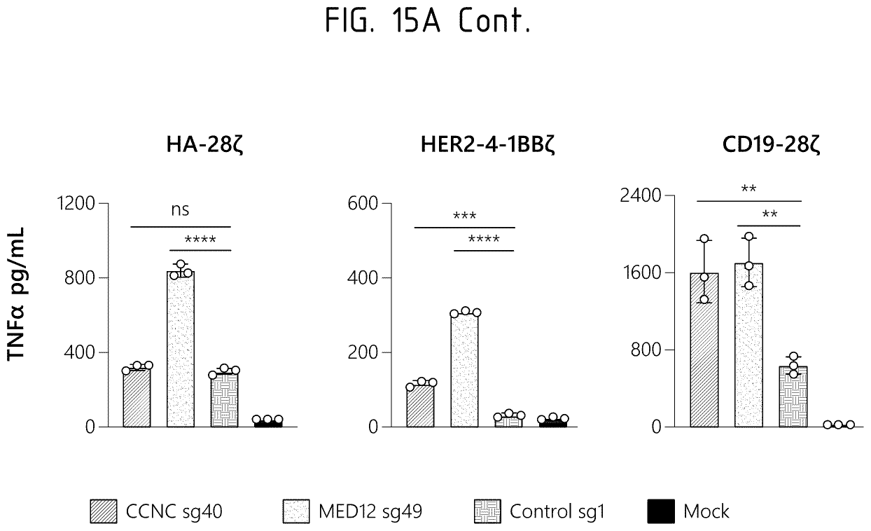

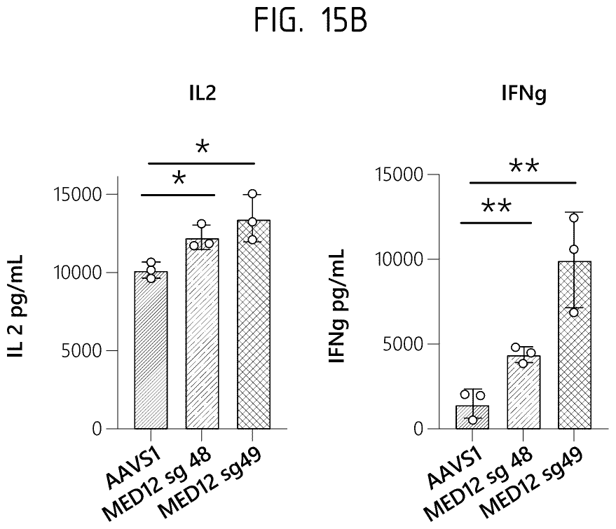

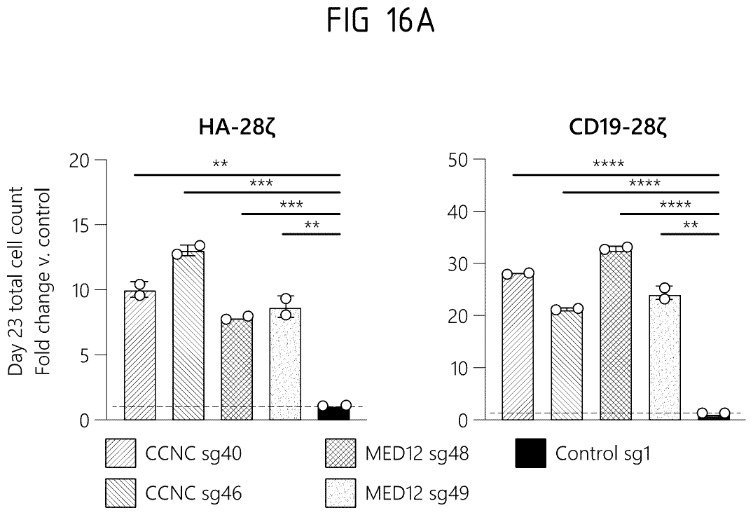

While the experimental data presented in Example 5 above validates the results of the CRISPR screen and indicates that MED12-null and CCNC-null CAR-T cells have 5-10 fold more expansion over 20 days in culture. The experimental data presented in Example 6 demonstrates that MED12-null and CCNC-null CAR-T cells still rely on IL-2 for survival, indicating these T cells are not transformed into cancer cells. This is an important observation since MED12 and CCNC mutations are implicated in some types of cancer. CCNC in particular is described as a tumor suppressor gene, but this experiment demonstrates that loss of CCNC alone was not sufficient for transformation. Additional experiments were performed to demonstrate that loss of MED12 or CCNC increases cytokine production in CAR-T cells or non-transduced T cells. In these experiments, 5×10⁴ T cells and 5×10⁴ tumor cells were co-cultured in 250 μl media without IL-2 in round bottom 96 well plates for 24 hours. Culture supernatants were collected following 24 hour co-culture with tumor cells and cytokines were quantified by ELISA (see, e.g., FIG. 15A) or CD3/CD28 activation beads (see, e.g., FIG. 15B). IL-2 and IFNγ were detected with the ELISA MAX kit (Biolegend) and TNF-alpha was detected with the Quantikine kit (R&D Systems). The experiments described in FIGS. 15A-15B are also described in Example 3 above.

### Example 7

### MED12-Null and CCNC-Null CD19-28ζ CAR-T Cells Demonstrate Increased Tumor Clearance In Vivo

This Example describes the results of experiments illustrating that MED12-null and CCNC-null CD19-28ζ CAR-T cells demonstrate increased tumor clearance in vivo.

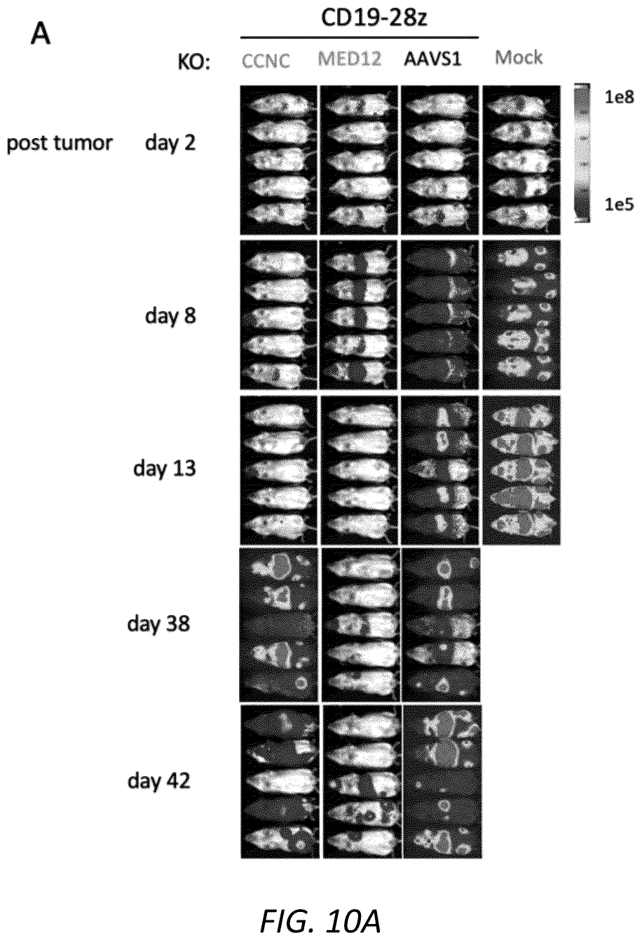

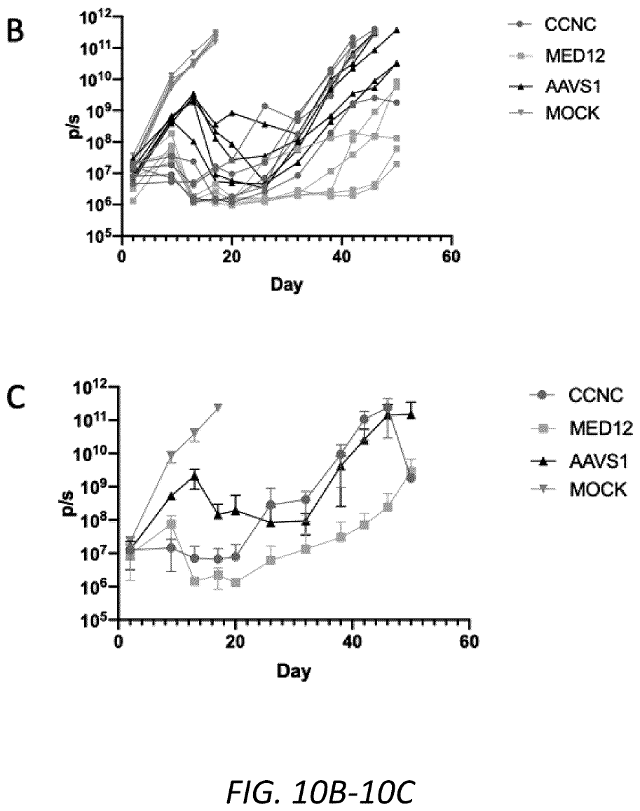

In these experiments, approximately 1 million NALM6 cells expressing a luciferase transgene were infused on Day 0, and 250,000 CAR-T cells or mock transduced T cells were infused on Day 3. Tumor burden was monitored by bioluminescence imaging using the SII Lago (Spectral Instruments Imaging) and photons per second (p/s) were quantified with Aura Imaging software. FIG. 10A shows 30 second exposures, whereas shorter exposures were used for quantification to avoid saturation (FIGS. 10B and 10C). Values for individual mice are shown in B and averages are show in C. Error bars depict standard deviation 1. It was observed that MED12-null and CCNC-null CD19-28ζ CAR-T cells demonstrate increased tumor clearance in vivo.

Remarkably, it was observed inn Example 7 that MED12-null and CCNC-null CAR-T cells exhibited dramatic and equal tumor clearance at early time points. However, by Day 42, mice treated with CCNC-null CAR-T cells had noticeably more tumor burden that those treated with CCNC-null CAR-T cells, indicating that loss of MED12 may be more tolerated by T cells over several weeks in vivo.

### Example 8

### MED12-Null and CCNC-Null CD19-28ζCAR-T Cells Demonstrate Increased Expansion In Vivo

This Example describes the results of experiments illustrating that MED12-null and CCNC-null CD19-28ζ CAR-T cells demonstrate increased expansion in vivo.

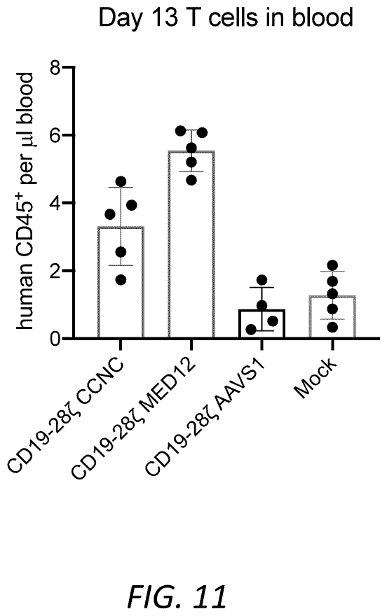

In these experiments, blood was collected from mice (as shown in FIG. 10 above) ten days after T cell infusion. Blood was mixed with an equal volume of CountBright Absolute Counting Beads (Invitrogen), stained for human CD45, and red blood cells were lysed with BD FACS Lysing Solution (BD). Human CD45+ cells were quantified by flow cytometry. As shown in FIG. 11, it was observed that MED12-null and CCNC-null CD19-28ζ CAR-T cells demonstrate increased tumor clearance in vivo.

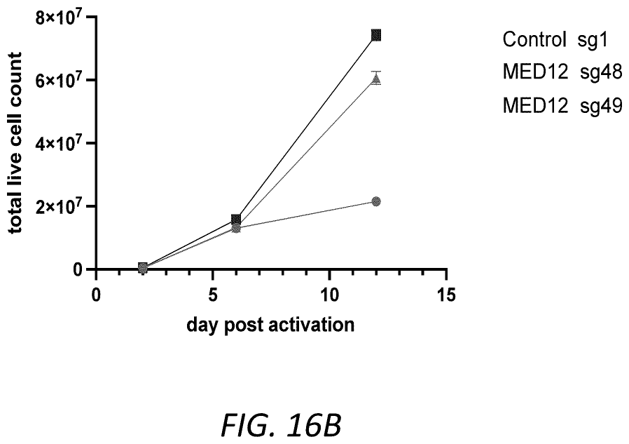

Additional experiments were performed to demonstrate that loss of MED12 increases expansion of CAR-T cells and T cells that do not express a CAR. In these experiments, CD4 and CD8 T cells were isolated from buffy coats and activated with anti-CD3/CD28 beads. Cells were CRISPR edited on day 3 and cultured with IL-2 until day 23 (see, e.g., FIG. 16A) or day 12 (see, e.g. FIG. 16B). In these experiments, cell counts and viability measurements were obtained using the Cellaca Mx Automated Cell Counter (Nexcelom). Cells were stained with acridine orange and propidium iodide to assess viability. A portion of the culture volume was discarded at each passage and the fraction of cells maintained in culture was used to calculate total cell counts.

Without being bound to any particular, theory, the data shown in Example 8 provides an explanation for why tumor clearance is enhanced. The increased number of MED12-null and CCNC-null CAR-T cells can account for decreased tumor burned at this timepoint. Additionally, this example is especially relevant because no T cell supporting cytokines such as IL-2 or IL-7 were administered. The increased production of IL-2 by these cells likely explains the increase in proliferation in vivo.

### Example 9

### MED12-Null and CCNC-Null CD19-28ζ CAR-T Cells Increase Survival Benefit of CAR-T Cell Treatment

This Example describes the results of experiments illustrating that MED12-null and CCNC-null CD19-28ζ CAR-T cells increase survival benefit of CAR-T cell treatment.

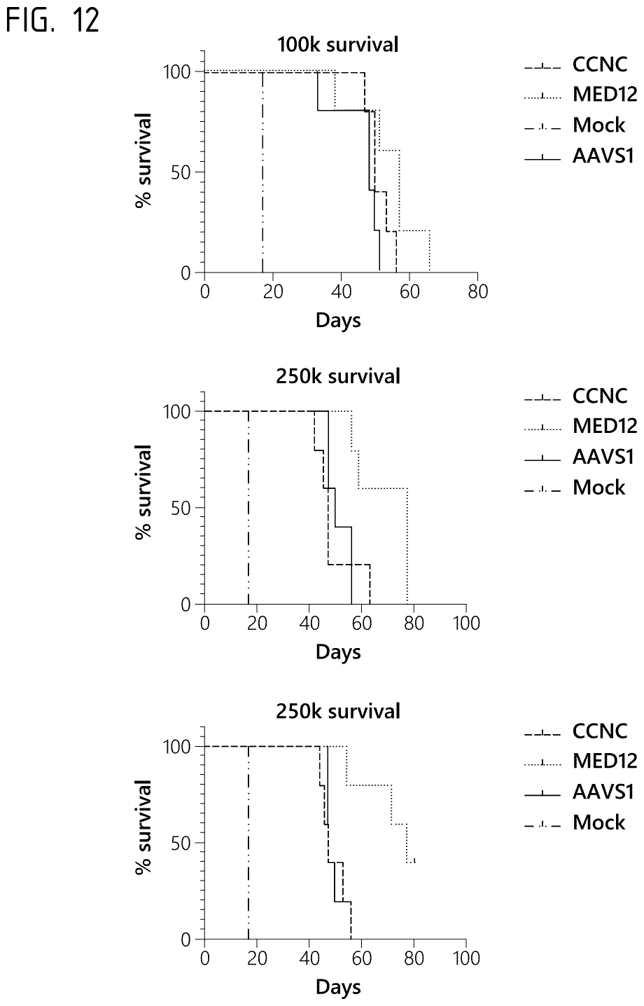

In these experiments, mice were infused with 1 million NALM6 tumor cells and treated with 1×10⁵, 2.5×10⁵, or 5×10⁵ T cells on Day 3. As shown in FIG. 12, MED12-null CAR-T cells increased survival, while CCNC-null CAR-T cells were equivalent to AAVS1-null CAR-T cells.

The experimental data presented in Examples 9 and 10 indicate that MED12-null and CCNC-null CAR-T cells are effective at slowing the progression of both liquid and solid tumors. Additionally, these examples show the effects of loss of MED12/CCNC are generalizable to CARs that target different antigens and utilize different co-stimulatory domains. This results suggests this strategy is applicable to all CAR-T cell therapeutics.

### Example 10

### MED12-Null and CCNC-Null HER2-4-1BBζ CAR-T Cells Decrease Solid Tumor Growth

This Example describes the results of experiments illustrating that MED12-null and CCNC-null HER2-4-1BBζ CAR-T cells decrease solid tumor growth and increase survival benefit of CAR-T cell treatment.

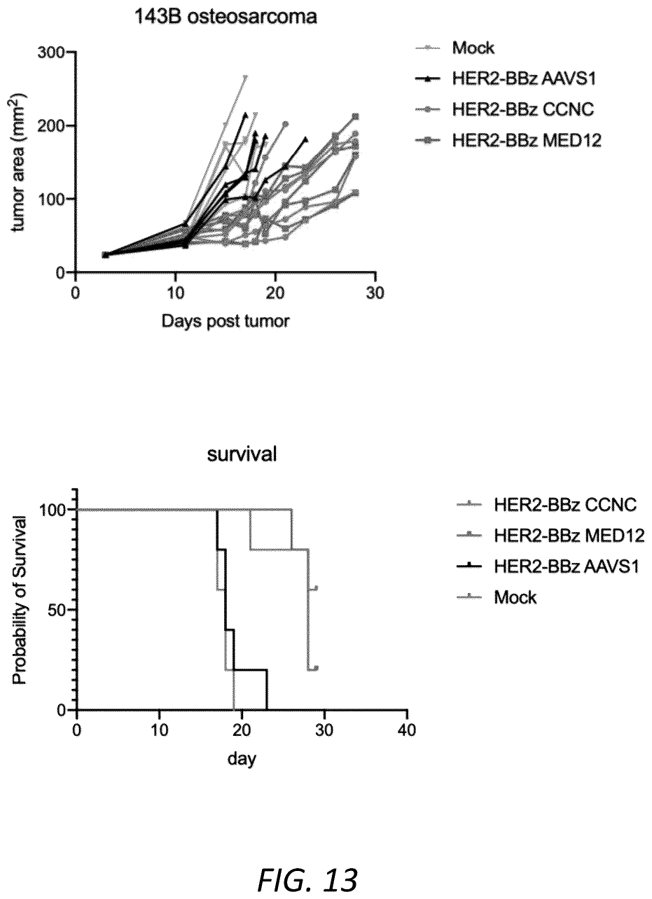

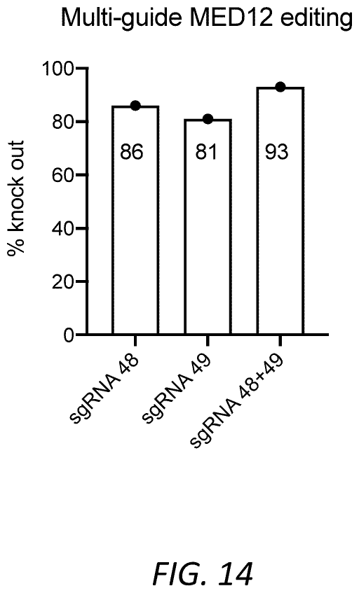

In these experiments, approximately 1 million 143B osteosarcoma cells were injected intramuscular on Day 0 and 5 million CAR-T cells or mock-transduced T cells were infused on day 4. Solid tumors were measured by calipers. Mice were euthanized when tumor diameter was ≥17 mm. As shown in FIG. 13, it was observed that MED12-null and CCNC-null HER2-4-1BBζ CAR-T cells decrease solid tumor growth (FIG. 1) and increase survival benefit of CAR-T cell treatment (FIG. 13B).

### Example 11

### Gene Deletion Frequency is Increased by Targeting 2 Cut Sites in MED12 Exon 2

This Example describes the results of experiments illustrating that gene deletion frequency is increased by targeting 2 cut sites in MED12 exon 2.

In these experiments, Alt-R® S.p. Cas9 Nuclease V3 (IDT) was diluted to 5 mg/mL in Duplex Buffer (IDT). sgRNAs (Synthego) were resuspended at 100 μM in TE buffer. 1 μL CAS9 and 1 μL sgRNA was combined and incubated 30 minutes at room temperature. For the two guide condition, 0.5 μl of each sgRNA was added. 2 million T cells were resuspend in 18 μL P3 buffer, mixed with CAS9, and pulsed with protocol EO115 using the P3 Primary Cell 4D-Nucleofector™ S Kit and 4D-Nucleofector™ System (Lonza). Cells were recovered immediately with 100 μL warm media, then transferred to 1 mL warm media for 6 hours prior to transduction with CAR. Editing was performed on day 3 and gene deletion was assessed on day 11.

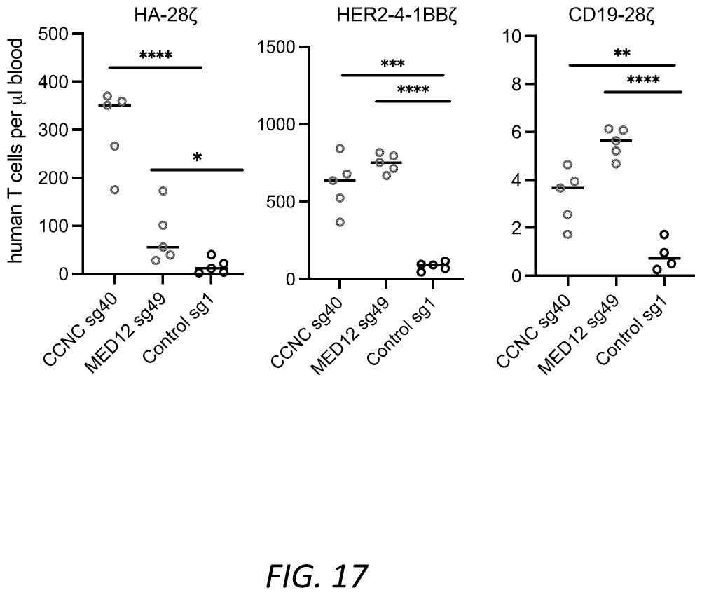

Additional experiments were performed to demonstrate that loss of MED12 or CCNC increases expansion in vivo. The frequency of T cells in peripheral blood was assessed 10 days after infusion into tumor bearing mice (see, e.g., FIG. 17). In these experiments, blood was collected from the retro-orbital sinus into Microvette blood collection tubes with EDTA (Fisher Scientific). Whole blood was labeled with anti-CD45 (HI30, ThermoFisher) and red blood cells were lysed with FACS Lysing Solution (BD) according to manufacturer's instructions. Samples were mixed with CountBright Absolute Counting beads (ThermoFisher) prior to flow cytometry analysis.

Experimental data presented in Example 11 exemplifies a method of efficiently optimizing deletion of MED12 with high efficiency. Typical CRISPR modification protocols include the use of one guide RNA. The method described in this Example included the use of two guides spaced approximately 50 bp apart. This approach resulted in a 47-bp indel that was generated at higher frequency than the 1-bp indel mutation generated by use of a single guide.

### Example 12

Treatment with CCNC-Null HER2-4-1BBζ CAR-T Cells Reduces Tumor Area and Increases Survival

This Example describes the results of experiments illustrating that treatment with CCNC-null HER2-4-1BBζ (CAR T cells reduces tumor area and increases survival of CAR-treated mice.

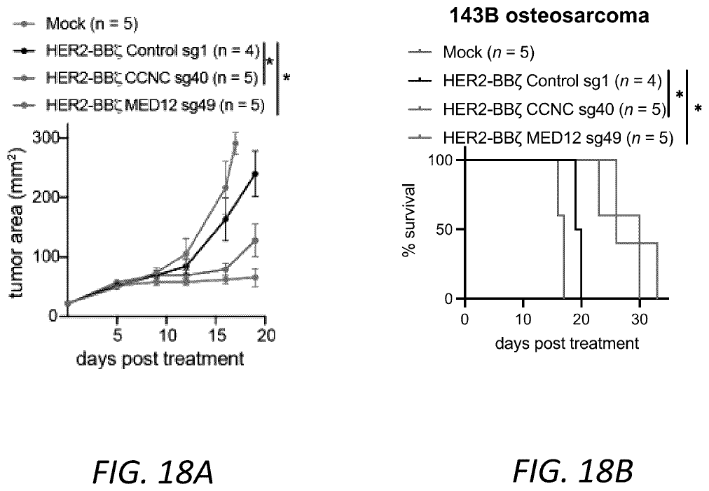

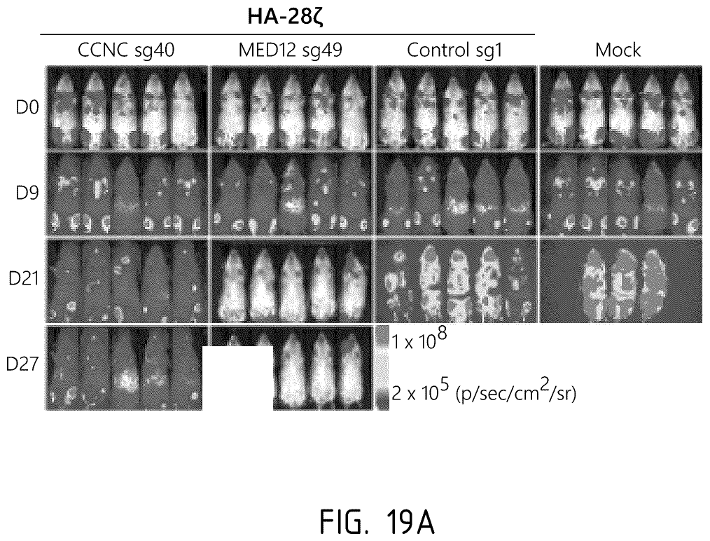

In these experiments, the tumor area of NSG mice was injected intramuscularly with 1×10⁶ 143B osteosarcoma cells and treated 4 days later with 5×10⁶ mock or CCNC⁻ or MED12-null HER2-4-1BBζ CAR-T cells. Tumor area was measured by caliper. Two-way ANOVA test with Dunnett's multiple comparison test. * P<0.01. As shown in FIG. 18A, the tumor area was lowest for mice treated with CCNC-null HER2-4-1BBζ CAR-T cells. FIG. 18B shows the percent survival for CAR-treated mice in FIG. 18A. Survival curves were compared with the Log-rank Mantel-Cox test. * P<0.01.

### Cell Lines:

NALM-6 leukemia cells and 143B osteosarcoma cells were obtained from American Type Culture Collection. Cell lines were stably transduced with GFP and firefly luciferase. Nalm6-GD2 was engineered to stably express GD2 synthase and GD3 synthase to obtain surface expression of GD2 disialoganglioside. Single cell clones were selected for high expression of GFP, luciferase, and GD2. Cell lines were maintained in RPMI (Gibco) supplemented with 10 mM HEPES, 10% FBS, and 1× penicillin-streptomycin-glutamine supplement (Gibco).

### Mice:

Immunocompromised NOD scid IL2Rgamma^{null} (NSG) mice were purchased from JAX and bred in-house under sterile conditions. Mice were monitored daily. Care and treatments were in compliance with Stanford University standard protocols. Leukemia cells and CAR-T cells were administered via intravenous injection. 143B osteosarcoma cells were administered by intramuscular injection. For some experiments, tumor burden was assessed prior to treatment and mice were randomized to groups to ensure equal tumor burden between treatment groups. Time of treatment and dosing is indicated in the figure. Researchers were blinded during administration of T cells. Leukemia progression was monitored using the Lago SII (Spectral Instruments Imaging). Quantification of bioluminescence was performed with Aura software (Spectral Instruments Imaging). Solid tumor progression was followed using caliper measurements of the injected leg area. Researchers were also blinded to the treatment groups during solid tumor measurements. Mice were euthanized upon manifestation of paralysis, impaired mobility, poor body condition (score of BC2−), or when tumor diameter exceed 17 mm. Sample sizes of 5 mice per group were selected based on previous experience with these models. All experiments were repeated twice with different donors, and donors used for in vivo experiments were different from the screening experiments.

### Example 13

Treatment with CCNC-Null HER2-4-1BBζ CAR-T Cells Increases Survival

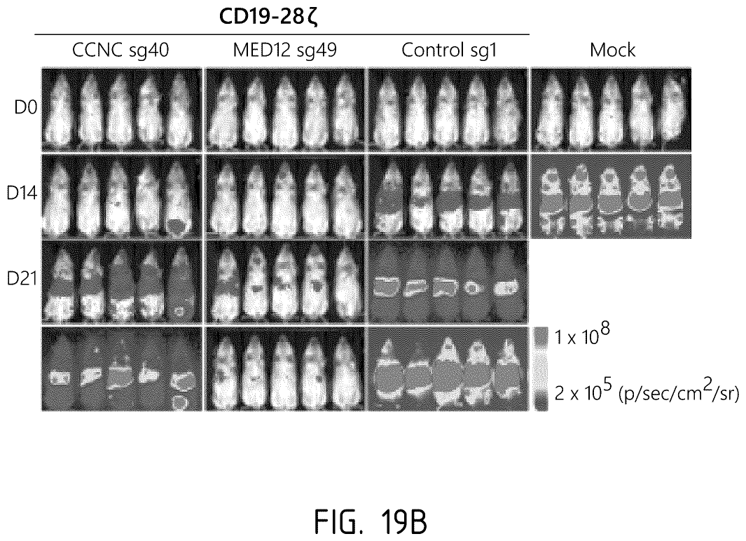

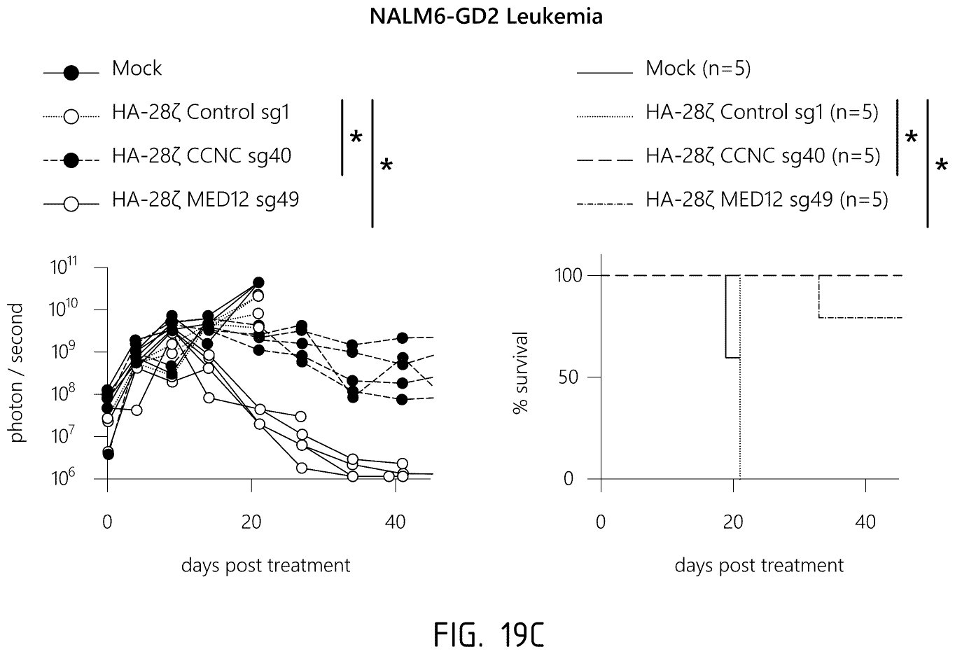

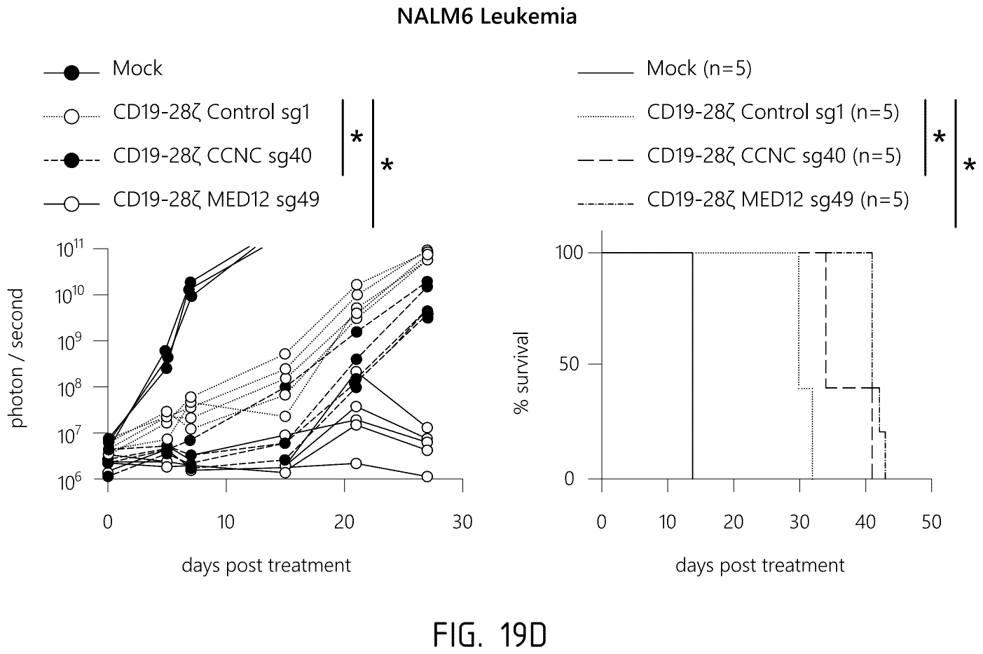

This Example describes the results of experiments illustrating that survival of CAR-treated mice is increased after treatment with CCNC-null HER2-4-1BBζ CAR-T cells. FIGS. 19A-19B show survival of CAR-treated mice. The number of mice per group is indicated in the figure legend. Tumor growth was monitored by bioluminescent imaging. Two-way ANOVA test with Dunnett's multiple comparison test. * P<0.01.

In experiments, NSG mice were injected intravenously with 1.0×10⁶ NALM6-GD2-Luc leukemia cells and treated with 2.0×10⁵ mock or CCNC- or MED12-null HA-28ζ CAR-T cells 9 days after tumor infusion (n=5 mice) (see, e.g., FIG. 19C). In other experiments, NSG mice were injected intravenously with 1.0×10⁶ NALM6-Luc leukemia and treated with 2.5×10⁵ mock or CCNC-null or MED12-null CD19-28ζ CAR-T cells 3 days after tumor infusion (n=5 mice) (see, e.g., FIG. 19D). Additional details of cell lines and mice used in these experiments are provided in Example 12 above.

### Example 14

Loss of MED12 increases expression of IL2RA

This Example describes the results of experiments illustrating that the loss of MED12 increases expression of IL2RA in T cells. In experiments, cells were CRISPR edited on day 3 post activation and subsequently transduced with CD19-28z CAR. Expression of IL2RA was assessed on day 15 post activation by flow cytometry. It was observed that MED12-deficient cells manifested an enhanced effector phenotype. Through ATAC-seq analysis, the observed enhancement of effector phenotype can be attributed to increased activity of the transcription factor STAT5, which is downstream of IL2RA. Therefore, without being bound to any particular theory, it is expected that IL2RA expression is an important phenotypic characteristic of MED12-deficient CAR-T cells. It is notable that elevated IL2RA expression in the absence of MED12 was found in both unmodified T cells and CAR-T cells, indicating that the mechanism of MED12 loss is not dependent on the presence of CAR.

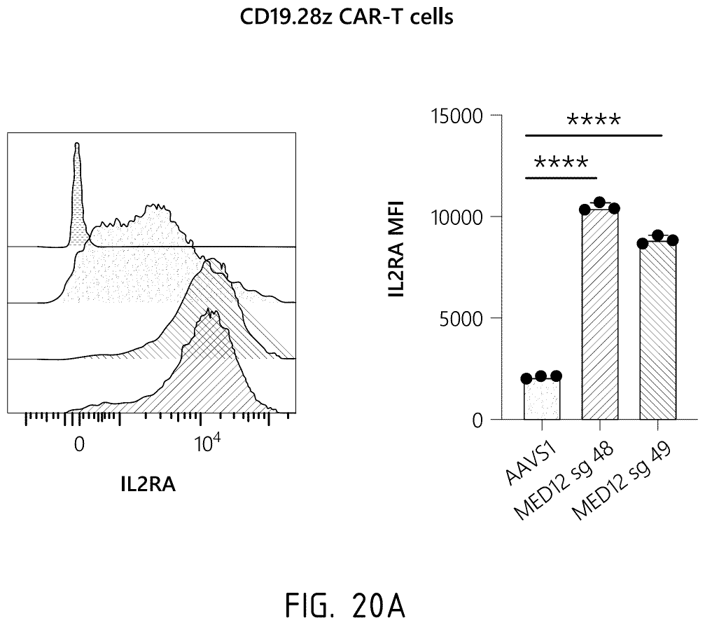

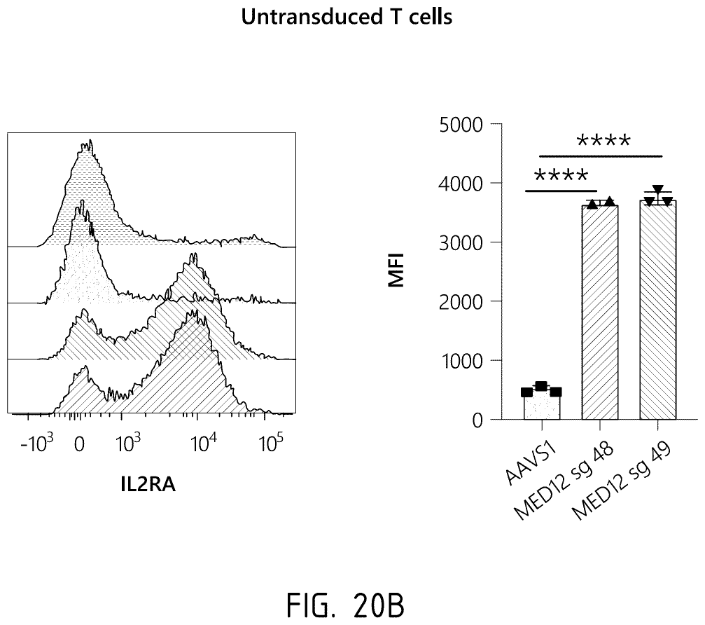

FIGS. 20A and 20B graphically summarize these experiments. FIG. 20A shows results for T cells expressing a CD19-28z CAR construct, while FIG. 20B shows results for untransduced T cells. In these experiments, T cells were washed in FACS buffer (DPBS no calcium, no magnesium (Gibco) with 2% FBS). Cells were incubated on ice in FACS buffer with antibodies specific for cell surface markers for twenty minutes. Cells were washed in FACS buffer and analyzed on an LSRFortessa (BD) with BD FACSDiva software.

### Example 15

### Loss of MED12 Increases Effector Memory T Cell Phenotype

This Example describes the results of experiments illustrating that the loss of MED12 increases effector memory T cell phenotype (CCR7 low, CD45RO high).

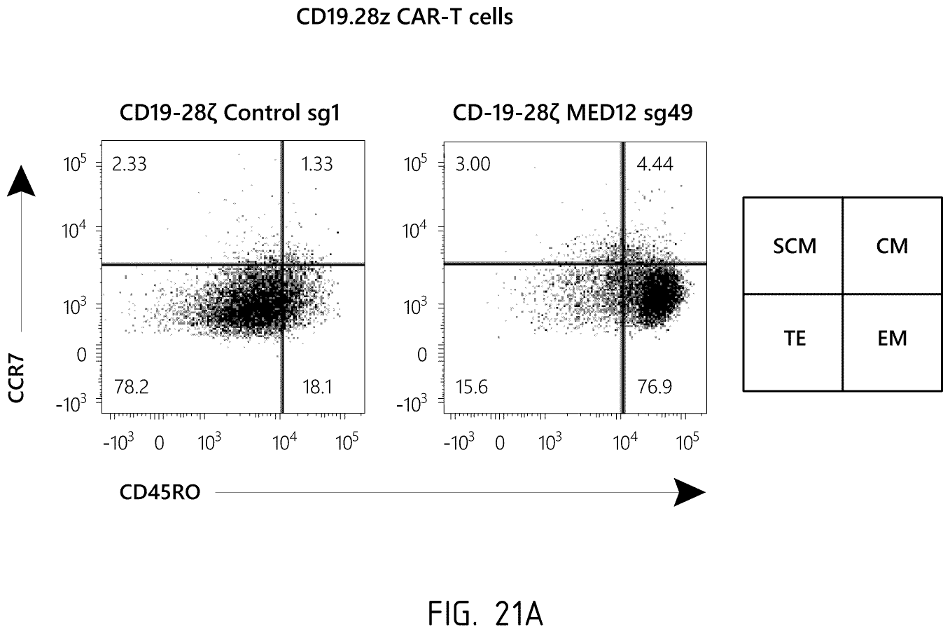

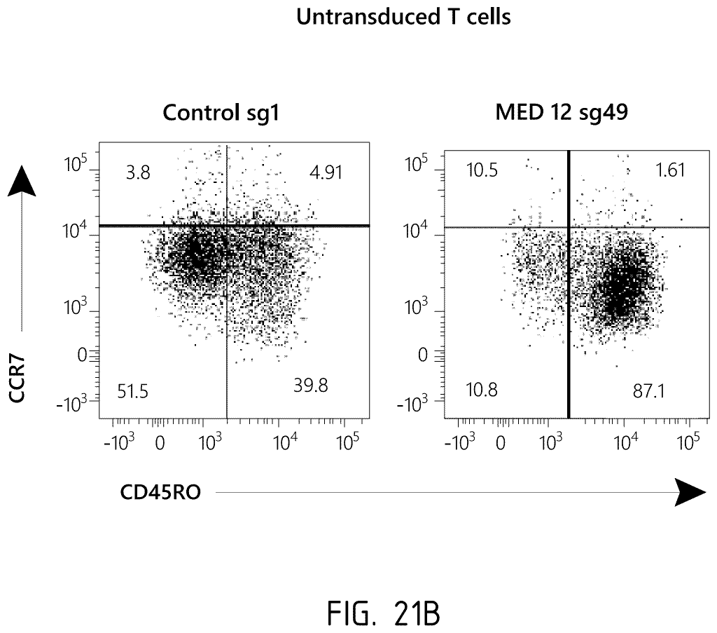

In these experiments, cells were assessed by flow cytometry 22 days post activation. FIG. 21A shows the cytometry results for CD19-28z CAR-T cells, where the following cell types were gated: stem cell-like memory T cells (SCM), central memory T cells (CM), effector memory T cells (EM), and terminally differentiated T cells (TE). FIG. 21B shows the results of a parallel experiments performed on for non-transduced T cells.

### Example 16

### Increased Oxygen Consumption and Extracellular Acidification Rates in MED12 Deficient CD19-28z CAR-T Cells

This Example describes the results of experiments performed to illustrate that a deficiency in MED12 increases the oxygen consumption rate and the extracellular acidification rate in CD19-28z CAR-T cells 15 days post activation. Without being bound to any particular theory, an increase in metabolic activity, as indicated by elevated oxygen consumption and extracellular acidification, can be considered as a feature of effector T cells which is in turn consistent with the hypothesis that loss of MED12 elicits an enhanced effector phenotype.

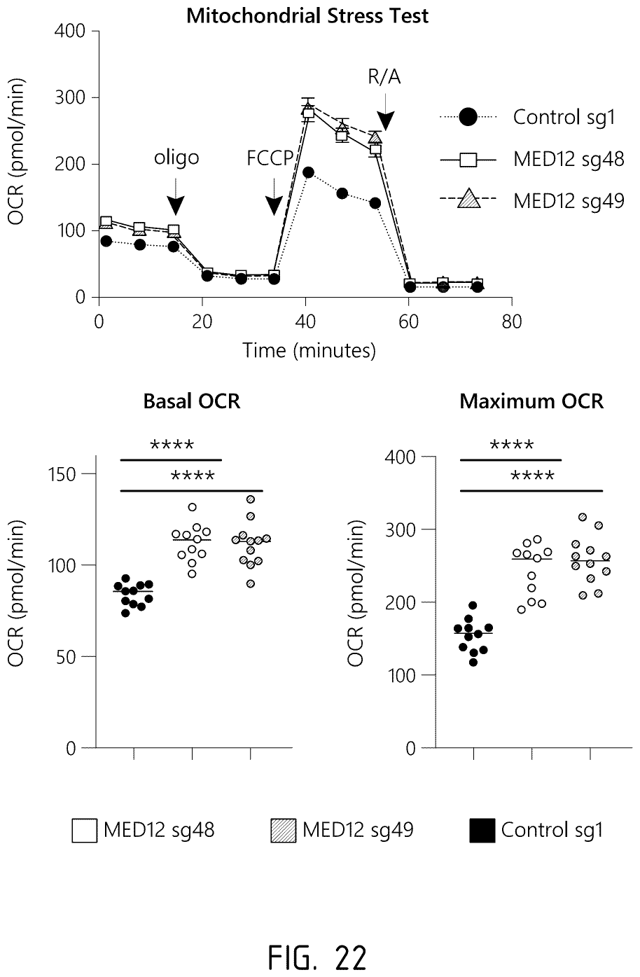

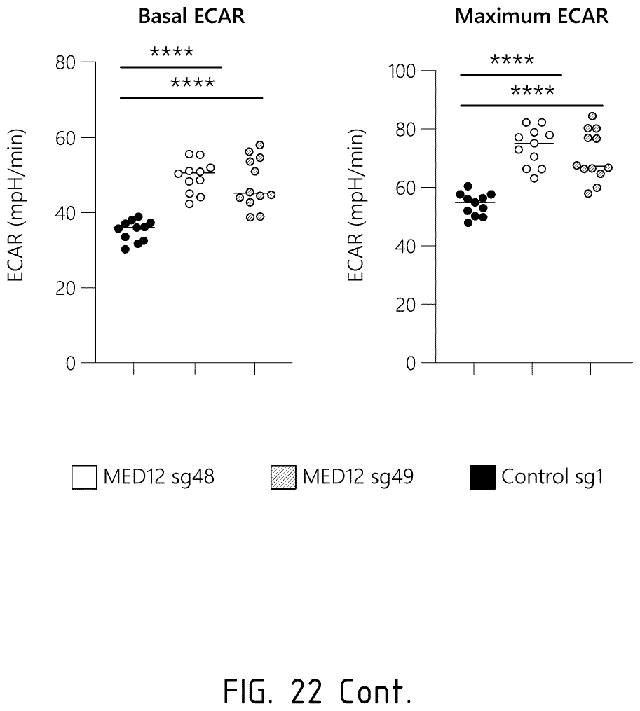

The mitochondrial stress test was performed using the Seahorse Assay (Agilent) according to manufacturer's instructions. FIG. 22 illustrates results of the mitochondrial stress test. In these experiments, metabolic analysis was carried out using Seahorse Bioscience Analyzer XFe96. Briefly, 2×10⁶ cells were resuspended in XF assay media supplemented with 25 mM glucose, 2 mM glutamine and 1 mM sodium pyruvate and plated on a Cell-Tak (Corning)-coated microplate allowing the adhesion of CAR T cells. Mitochondrial stress and glycolytic parameters were measured via oxygen consumption rate (OCR) (pmol/min) and extracellular acidification rate (ECAR) (mpH/min), respectively, with use of real-time injections of oligomycin (1.5 μM), carbonyl cyanide-4 (trifluoromethoxy)phenylhydrazone (FCCP; 1 μM) and rotenone and antimycin (both 1 μM). Respiratory parameters were calculated following manufacturer's instructions (Seahorse Bioscience). All chemicals were purchased from Agilent unless stated otherwise.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

### Source of CRISPR Library Reagents

Morgens D W, Wainberg M, Boyle E A, Ursu O, Araya C L, Tsui C K, Haney M S, Hess G T, Han K, Jeng E E, Li A, Snyder M P, Greenleaf W J, Kundaje A, Bassik M C. Genome-scale measurement of off-target activity using Cas9 toxicity in high-throughput screens. Nat Commun. 2017 May 5; 8:15178.

### DNA Extraction and Sequencing Methods

Yau E H, Rana™. Next-Generation Sequencing of Genome-Wide CRISPR Screens. Methods Mol Biol. 2018; 1712:203-216.

### Source for Algorithm to Analyze CRISPR Screen

W. Li, H. Xu, T. Xiao, L. Cong, M. I. Love, F. Zhang, R. A. Irizarry, J. S. Liu, M. Brown, X. Liu, MAGeCK enables robust identification of essential genes from genome-scale CRISPR/Cas9 knockout screens, Genome Biol. 15 (2014) 554.

### ADDITIONAL REFERENCES

Morgans D. W. et al. Genome-scale measurement of off-target activity using Cas9 toxicity in high-throughput screens. Nat Commun. 2017 May 5; 8:15178.
Rose J. C. et al. Rapidly inducible Cas9 and DSB-ddPCR to probe editing kinetics. Nat Methods. 2017 September; 14(9):891-896.
Lynn R. C. et al. c-Jun overexpression in CAR T cells induces exhaustion resistance. Nature. 2019 December; 576(7786): 293-300.

1.-41. (canceled)

42. A method for generating an engineered immune cell with enhanced effector function, the method comprising introducing into the immune cell a nucleic acid and/or a polypeptide capable of reducing expression level of a mediator complex subunit in the immune cell.

43. The method of claim 42, wherein the mediator complex subunit is selected from the middle module subunits, the tail module subunits, and the cyclin-dependent-kinase 8 (CDK8) module subunits of the mediator complex.

44. The method of claim 42, wherein the mediator complex subunit is selected from the group consisting of CCNC, CDK8, CDK19, MED12, MED12L, MED13, MED13L, MED19, MED24, and MED26.

45. The method of claim 42, wherein the nucleic acid is incorporated into one or more of the following: (i) a guide RNA (gRNA) of a CRISPR/Cas genome editing system, (ii) a TALEN (transcription activator-like effector nuclease) genome editing system, (iii) a DNA-guided endonuclease genome editing with NgAgo (Natronobacterium gregoryi Argonaute), (iv) anti-sense nucleic acid molecule, (v) a double-stranded RNAi molecule, and (vi) a hairpin-RNA molecule capable of inducing suppression or degradation of mRNA.

46. The method of claim 42, wherein the nucleic acid comprises a polynucleotide sequence having sufficient sequence complementarity to a target sequence within an endogenous genomic locus encoding the mediator complex subunit.

47. The method of claim 42 wherein the immune cell is T lymphocyte, a natural killer (NK) cell, or a macrophage.

48. The method of claim 47, wherein the T lymphocyte is:
(a) a CD8+T cytotoxic lymphocyte cell selected from the group consisting of naïve CD8+ T cells, central memory CD8+ T cells, effector memory CD8+ T cells, effector CD8+ T cells, CD8+ stem memory T cells, bulk CD8+ T cells; or
(b) a CD4+T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells.

49. The method of claim 42, wherein further comprising introducing into the immune cell one or more recombinant immune receptors.

50. The method of claim 49, wherein the one or more recombinant immune receptors comprises a chimeric antigen receptor (CAR) and/or a T cell receptor (TCR).

51. An engineered immune cell produced by a method according to claim 42.

52. The engineered immune cell of claim 51, wherein the immune cell is in vitro, ex vivo, or in vivo.

53. The engineered immune cell of claim 51, wherein the immune cell is an exhausted immune cell or a non-exhausted immune cell.

54. A pharmaceutical composition comprising a pharmaceutically acceptable excipient and an engineered immune cell according to claim 51.

55. The pharmaceutical composition of claim 54, wherein the mediator complex subunit is selected from the group consisting of CCNC, CDK8, CDK19, MED12, MED12L, MED13, MED13L, MED19, MED24, and MED26.

56. The pharmaceutical composition of claim 54, wherein the composition comprises the nucleic acid is encapsulated in a viral capsid, a liposome, or a lipid nanoparticle (LNP).

57. A method for treating a health condition in a subject in need thereof, the method comprising administering to the subject a composition comprising:
a) an engineered immune cell according to claim 51; and/or
b) a pharmaceutical composition comprising an engineered immune cell of (a).

58. The method of claim 57, wherein the health condition is a proliferative disease, an autoimmune disease, or an infection.

59. The method of claim 57, wherein the administered composition confers an enhanced effector function selected from the group consisting of growth rate, cytokine production, target cell inhibition, macrophage activation, T cell activation, NK cell activation, and in vivo persistence.

60. The method of claim 59, wherein the enhanced effector function comprises one or more of the following: increased production of interferon gamma (INFY), interleukin-2 (IL-2), tumor-necrosis factor α (TNFα), increased effector memory T cell phenotype, and increased oxygen consumption and extracellular acidification rate.

61. A kit for the prevention and/or treatment of a condition in a subject in need thereof, the kit comprising:
a) an engineered immune cell according to claim 51; and/or
b) a pharmaceutical composition comprising an engineered immune cell of (a).
